(19) United States
(12) Patent Application Publication
(10) Pub. No.: US 2026/0256434 A1
(43) Pub. Date: Sep. 3, 2026

(54) COMPTON SCATTERING IMAGING BASED ON TIME-OF-FLIGHT OF PHOTONS

(71) Applicant: SOCPRA SCIENCES ET GÉNIE S.E.C., Sherbrooke (CA)

(72) Inventors: Réjean FONTAINE, Sherbrooke (CA); Yves BÉRUBÉ-LAUZIÈRE, Sherbrooke (CA); David GAUDREAULT, Québec (CA); Julien ROSSIGNOL, Sherbrooke (CA)

(21) Appl. No.: 19/648,838

(22) Filed: Apr. 15, 2026

Related U.S. Application Data
(63) Continuation-in-part of application No. PCT/CA2024/051359, filed on Oct. 16, 2024.
(60) Provisional application No. 63/591,372, filed on Oct. 18, 2023.

(51) Int. Cl. A61B 6/42; A61B 6/00

(52) U.S. Cl. CPC A61B 6/4241; A61B 6/483; A61B 6/5205

(57) ABSTRACT

Measured and selected scattered photons from an X-ray imaging acquisition are used to better characterize the X-rayed object and/or to generate an improved X-ray image combining ballistic and scattering information. Scattering-based X-ray images are generated using a measure of scattered photons from an X-ray absorption acquisition and are used to determine the scattering index of the scanned object. This information can be used to determine the nature of the scanned object and to better understand its structures. Such information is derived from a conventional X-ray absorption scan. Information provided by time-of-flight measurements of scattered (non-ballistic) photons allows to determine an ellipse of possible scattering positions, the shape of which is determined from the corresponding time-of-flight. The proposed method and apparatus are used to generate scattering images and to characterize additional properties of scanned objects, which can be combined or used complementarily to conventional X-ray absorption scans.

The present application is a bypass continuation in part (CIP) of the PCT application PCT/CA2024/051359 filed on Oct. 16, 2024 designating the United States, and claiming priority therefrom, which claims priority from U.S. provisional patent application No. 63/591,372 filed on Oct. 18, 2023, incorporated herein by reference.

### TECHNICAL FIELD

The present invention relates to X-ray imaging, more specifically to radiography and computed tomography (CT) imaging apparatus and methods.

### BACKGROUND

Many approaches have been proposed to reduce the dose deposited in a subject. It was shown in the PCT publication WO2020093140 published 14 May 2020, that improving the method of measuring the photons at the photon detector level in X-ray imaging, such as in radiography or computed tomography (CT) systems, could lead to a significant improvement of the quality of the X-ray imaging modality using a smaller overall X-ray dose. More specifically, it showed that the X-ray imaging system comprising a pulsed X-ray source with a time-sensitive X-ray detector to provide a specific measure of ballistic photons with a reduction (i.e., filtering out) of scattered photons produces a comparable contrast-to-noise X-ray image using significantly less radiation exposure than conventional X-ray images, notably about half of the radiation. In this previously proposed method, the scattered photons that were filtered out were simply discarded to focus on generating an optimized X-ray image reconstituted from the measured ballistic photons, i.e., an absorption image representing the various absorption coefficients of the various tissues, and thus provide structural information on the subject (bone, fat, muscle, water, air, etc.).

Calvert et. al (Feasibility Study of Time-of-Flight Compton Scatter Imaging Using Picosecond Length X-ray Pulses, IEEE transactions on nuclear science, vol 61, no. 6, December 2014) completed simulations and experiments attempting to use the time-of-flight of X-ray photons to reconstruct the position of scatter of high energy X-ray photons which hinted that it may lead to the reconstruction of scatter points to provide three-dimensional information of the object under inspection enabling a low detail three-dimensional image. However, these simulations and experiments were limited to scattered photons and do not simultaneously acquire ballistic and scattered photons. Therefore, in this specific case, the scattered image cannot be corrected with the absorption image obtained with ballistic photons. Moreover, the setup does not allow for the acquisition of medical images since the timescale used is in the nanosecond (ns) range and pixel density and size is very low.

It would be useful to develop a method and/or apparatus that would allow for the use of scattered photons measurement and absorption measurement to generate a more precise X-ray image that would extract further information from these measurements. It will be appreciated that it would be further useful, efficient and medically preferable (i.e., to minimize/reduce the patient's X-ray dose) to acquire these measurements with the same X-ray measurement/acquisition. However, the current state of the art is silent on such a method and/or apparatus.

### SUMMARY

A broad aspect of the present disclosure enabled the use of the filtered out scattered photons from an X-ray imaging acquisition (e.g., absorption imaging) to better characterize the X-rayed object or to generate a scattering X-ray image, which can be complementary to the absorption images. It was demonstrated that an absorption-based X-ray measurement/acquisition originating from an X-ray pulse can be used to simultaneously reconstruct/generate an absorption-based X-ray image and a scattering-based X-ray image. Namely, the scattering-based X-ray image can be generated using a measurement of scattered photons extracted or selected from the same absorption-based X-ray acquisition, where the scattering index and the material of an object/phantom can be determined based on the measured scattered photons. Time-of-flight measurements of scattered (non-ballistic) photons allow to determine shape/dimensions of an ellipse of possible scattering positions, which can be used to determine/extract additional information about the scanned objects or their structures.

The method can use the scattered photons, normally unused or undesired in current scan imaging (e.g., imaging by absorption), to acquire and/or determine complementary information about the scanned object, such as information about the nature of some of its structures. Such information can be derived from, combined with or used complementarily to an absorption image that can be obtained with a same scan (same acquisitions) used to measure the scattered photons, for example. Generating an absorption image based on time-of-flight measurements can be achieved, for example, with the method described in the US publication US 2021/0369222 A1.

Another broad aspect of the present disclosure is developed a novel method and an apparatus for using this method that can utilize and analyze a measurement of diffused/scattered X photons to generate scattering images, notably to characterize some properties of a scanned object to give better diagnostics by quantifying various characteristics of materials related to scattering and composing the scanned object.

Another broad aspect of the present disclosure is an X-ray imaging apparatus comprising: an input interface for receiving time-dependent X-photon detection data; a processor; and non-transitory memory storing program code that, when executed by the processor, causes the processor to: A) receive the time-dependent X-photon detection data that comprises time-of-flights and detection locations of photons from a single X-ray measurement; B) identify, for a plurality of the detection locations, detected scattered photons having a single scattering event and detected ballistic photons from the time-of-flights; C) associate possible locations of a single scattering event for each of the detected scattered photons having the single scattering event, wherein the possible locations comprise at least one portion of an ellipsoid surface of possible positions of the single scattering event; D) use at least a plurality of the possible locations of the single scattering event to generate a scattering representation; and E) use at least a plurality of the detection locations of the detected ballistic photons to generate an absorption representation.

In some embodiments, the program code further causes the processor to use the absorption representation to correct the scattering representation.

In some embodiments, the program code causes the processor to generate a corrected scattering representation using the at least a plurality of the possible locations of the single scattering and the detection locations of the detected ballistic photons.

In some embodiments, the X-photon detection data comprises a plurality of time-of-flights and a corresponding detection location for each one of the time-of-flights.

In some embodiments, the X-photon detection data comprises a distribution of the detected ballistic photons and the scattered photons as a function of a corresponding time-of-flight for each of the detection locations.

In some embodiments, the identifying of the detected scattered photons having the single scattering event from the time-of-flights comprises using at least one of: A) a time-of-flight threshold; B) the time-of-flight threshold and a first time-of-flight cut-off; and C) a shape of the distribution of the detected ballistic photons and the scattered photons as a function of a corresponding time-of-flight.

In some embodiments, the identifying of the detected ballistic photons from the time-of-flights comprises using at least one of: A) a second time-of-flight cut-off; and B) the shape of the distribution of the detected ballistic photons and the scattered photons as a function of a corresponding time-of-flight.

In some embodiments, the distribution of the detected ballistic photons and the scattered photons as a function of a corresponding time-of-flight is a temporal point-spread function.

In some embodiments, the second time-of-flight cut-off is a time-of-flight threshold.

In some embodiments, the time-of-flight cut-off and/or the time-of-flight threshold depends on a corresponding detection location.

In some embodiments, the at least one portion of the ellipsoid surface is a portion of the ellipsoid surface excluding portions of the ellipsoid surface outside of a working region of the X-ray measurement.

In some embodiments, the scattering representation is a superimposition of the possible locations.

In some embodiments, the scattering representation is a 2D image.

In some embodiments, an electron density of at least one pixel of the scattering representation is determined.

In some embodiments, the scattering representation is a 3D image.

In some embodiments, an electron density of at least one voxel of the scattering representation is determined.

In some embodiments, the electron density is used to determine a corresponding material.

In some embodiments, the scattering representation and the absorption representation are combined in an X-ray representation.

In some embodiments, at least one pixel/voxel of the X-ray representation is color coded as a function of the electron density and/or the corresponding material.

In some embodiments, the apparatus further comprises a pulsed X-ray source having a control signal, and a time-sensitive X-ray detector for generating the time-dependent X-photon detection data.

In some embodiments, the pulsed X-ray source comprises a high-voltage source, electrodes connected to the high-voltage source for accelerating electrons, and an X-ray emitting target material arranged to receive the electrons following acceleration by the electrodes so as to produce a pulse of X-rays.

In some embodiments, the pulsed X-ray source comprises deflection electrodes for steering the electrons accelerated by the electrodes connected to the high-voltage source to controllably hit the X-ray emitting target material.

In some embodiments, the pulsed X-ray source comprises a pulsed laser source responsive to the control signal, and a photoelectric material arranged to receive a light pulse from the pulsed laser source and to emit a burst of electrons in response thereto, wherein electrodes are arranged to accelerate the burst of electrons.

In some embodiments, the photoelectric material is at least a part of a cathode of the electrodes.

In some embodiments, the time-sensitive X-ray detector is responsive to a time window signal for enabling a detection of the time-sensitive X-ray detector during the time-of-flight threshold or the time-of-flight range or disabling the detection.

In some embodiments, the pulsed X-ray source produces a cone beam and the time-sensitive X-ray detector is arranged as a 2D array of detector cells.

In some embodiments, a rise time of a pulse emitted by the pulsed X-ray source is less than 0.15 nanoseconds, and a response time of a combination of the pulsed X-ray source and the time-sensitive X-ray detector is less than 0.9 nanoseconds, preferably less than 0.3 nanoseconds.

In some embodiments, the processor is further used for measuring an impulse response time of a combination of the pulsed X-ray source and the time-sensitive X-ray detector to obtain a measure of ballistic photons without an object or patient between the pulsed X-ray source and the time-sensitive X-ray detector, and to derive therefrom and store in memory a gate parameter for discriminate the detected scattered photons from a time-dependent X-photon detection signal of the time-sensitive X-ray detector.

In some embodiments, the X-ray pulse comprises photons having an energy of more than about 80 keV, preferably more than about 100 keV.

In some embodiments, the possible locations of the single scattering event are determined using back projection, filtered back projection, iterative method or artificial intelligence-based algorithms.

Another broad aspect of the present disclosure is a method of reconstructing a medical diagnostic image of a human patient comprising; providing a measurement comprising detection locations and times-of-flights of detected photons from an X-ray scan, wherein the detected photons comprise detected ballistic photons and detected scattered photons; using the detection location and the time-of-flight of each one of the detected scattered photons having a single scattering event to associate possible locations of the single scattering event; wherein each one of the possible locations comprises at least one portion of an ellipsoid surface of possible positions of the single scattering event of a corresponding one of the detected scattered photons; and generating a scattering representation of the region of interest using at least a plurality of the possible locations.

In some embodiments, the at least one portion of the ellipsoid surface is a portion of the ellipsoid surface comprised within a working region of the X-ray imaging apparatus.

In some embodiments, the method further comprises identifying the detected scattered photons having the single scattering event and the detected ballistic photons from the measurement based on at least one of: A) a time-of-flight threshold; B) the time-of-flight threshold and at least one time-of-flight cut-off; and C) a shape of a distribution of the detected ballistic photons and the scattered photons as a function of a corresponding the time-of-flight.

In some embodiments, the detection locations of the detected ballistic photons are used to generate an absorption representation.

In some embodiments, the generating comprises using the possible locations of the single scattering event and the detection locations of the detected ballistic photons to generate a corrected scattering representation.

In some embodiments, the scattering representation is used in combination with the absorption representation.

In some embodiments, an amount of radiation delivered to the patient is about 30% or less of an amount of radiation delivered to a same patient for continuous, polychromatic X-ray imaging of the region of interest using the given energy of X-rays.

In some embodiments, the possible locations of the single scattering event are determined using back projection, filtered back projection, iterative methods or artificial intelligence-based algorithms.

In some embodiments, the distribution of the detected ballistic photons and the scattered photons as a function of a corresponding the time-of-flight is a temporal point-spread function.

### BRIEF DESCRIPTION OF THE DRAWINGS

The invention will be better understood by way of the following detailed description of embodiments of the invention with reference to the appended drawings, in which:

FIG. 1A is a schematic drawing of a fan beam computed tomography setup.

FIG. 1B is a schematic drawing of a cone beam computed tomography setup.

FIG. 1C is an exemplary distribution of counts of measured X photons (ballistic vs scattered) as a function of time (time-of-flight) for the computed tomography setups, where the measurement is subsequent to the emission of a short X-ray pulse.

FIG. 1D is a block diagram of the possible steps required to refine the discrimination and selection of a measurement of scattered photons with a reduction of ballistic photons, or a measurement of ballistic photons with a reduction of scattered photons, by utilizing a temporal point-spread function (TPSF) analysis.

FIG. 1E is another block diagram of the possible steps required to refine the discrimination and selection of a measurement of scattered photons with a reduction of ballistic photons, or a measurement of ballistic photons with a reduction of scattered photons, by utilizing a time-of-flight distribution analysis.

FIG. 2A is a schematic drawing of the single 2D time-of-flight acquisition detecting a ballistic and a scattered X photon emitted by the source at a same time, passing through an object and arriving at the detector at two different times owing to their different paths.

FIG. 2B is a schematic drawing of the portion of a theoretical ellipse of possible positions of a given scattering event of a detected scattered X photon, with the event having taken place inside the object.

FIG. 2C is a schematic drawing of two theoretical ellipses of possible positions of two consecutive scattering events, detected by a same detector cell at different times, the events having taken place inside the object.

FIG. 2D shows a schematic drawing of an ellipse of possible positions of a given scattering event with various areas within its perimeter.

FIG. 2E defines the characteristics of an ellipse.

FIG. 3A is a schematic drawing of the single 2D measurement detecting various scattered X photons, with a 2D triangular working region and without any predefined object.

FIG. 3B shows the portions of both ellipses of FIG. 3A located in a field of view from an alternative orientation.

FIG. 3C shows multiple traces of the portions of ellipses of possible positions for various scattered X photons measured with a thin 2D detector during a single scan using a fan-shaped beam on a pixel grid located in a field of view from an alternative reconstruction perspective.

FIG. 3D shows the relationship between the detection time of an X-photon having various time-of-flights detected by a detector cell having a fixed position and the shape of the ellipse of possible scattering locations.

FIG. 4A shows a simulation result of a sum of ellipses of possible scattering positions for all scattered photons detected at n projection angles corresponding to different angular positions (360°/n) around the cylindrical object/phantom.

FIG. 4B shows the line profile across the horizontal line of the simulation result of FIG. 4A corresponding to the number of ellipses crossing at various positions along this horizontal line and superimposed.

FIG. 5A shows a schematic drawing of a phantom with inserts made of different materials used for the simulation of FIG. 5B.

FIG. 5B shows a simulation result of a sum of ellipses of possible scattering positions for all scattered photons detected at n projection angles corresponding to angular positions (360°/n) around the cylindrical phantoms illustrated in FIG. 5A.

FIG. 5C shows the line profiles across different white lines of the simulation result of FIG. 5B corresponding to the number of ellipses crossing at various positions along the lines depicted at the top-right corner of the Figure.

FIG. 5D shows a simulated absorption representation of a scan of cylindrical phantoms illustrated in FIG. 5A.

FIG. 6 shows a schematic drawing of an embodiment of the X-ray imaging apparatus.

FIG. 7 shows various steps that can be required to use the X-ray imaging apparatus.

FIG. 8A shows a drawing of a cross-section of a cone-shaped beam scanning an arrangement of various scattering and absorbing objects.

FIG. 8B shows a drawing of a scattering reconstruction of the scan of the setup illustrated in FIG. 8A.

FIG. 8C shows a drawing of an absorption representation of the scan of the setup illustrated in FIG. 8A superimposed on the scattering reconstruction of FIG. 8B.

FIG. 8D shows a schematic 2D drawing that illustrates how an ellipse of possible scattering locations of a detected scattered photon can be combined with the 2D results of an absorption measurement that detected two absorbing objects casting shadows of photons (reduction of photons density) in the scanned region in order to identify which part of the ellipsoid should be adjusted and corrected for such shadows.

FIG. 8E shows a schematic 2D drawing that illustrates how positions of a scattering event on the ellipsoid of possible scattering locations can be considered for adjusting and correcting for the probability of scattering as a function of the associated scattering angle.

FIG. 9 shows schematically photoelectric absorption and Compton scattering for bones and soft tissues as a function of X photon energy in the range from 30 to 120 keV.

FIG. 10A is a histogram of the number of photons measured experimentally with nothing between the source and the detectors according to the time between the source trigger and the detection which is fitted to a landau distribution.

FIG. 10B is a histogram of the number of photons measured experimentally with a 4 cm thick beam-blocker between the source and the detectors according to the time between the source trigger and the detection which is fitted to a landau distribution.

FIG. 11A is a schematic sectional side view of a laser-pulsed cathode side-window type of X-ray tube.

FIG. 11B is a schematic sectional side view of a carbon nanotubes (CNT) gated cathode side-window type of pulsed X-ray tube.

FIG. 12 shows a schematic representation of an imaging system comprising a pulsed X-ray source for measuring TPSF data, the computing device, at least one workstation (local or remote) and an optional database for storing the resulting X-ray data and images.

### DETAILED DESCRIPTION

In the present document, the term “object” is understood to mean an imaging phantom, a body, part of a body, including bones, muscles, fat, organs and blood vessels, a machined part, or any object permeable to X photons, etc.

In the present document, an ellipse is understood to be a subset or a particular part of an ellipsoid. An ellipse corresponds to a cross-section of an ellipsoid and a plane comprising the two ellipsoid's foci. Also when referring to an arc portion of an ellipse in 2D, it is implied that it would correspond to a region of the surface of an ellipsoid in 3D.

I will be appreciated that the term “representation”, in the present document, can include any suitable visual illustration/image/image-stack/hologram/etc. that may be generated and/or displayed in 2D or 3D and, optionally, with colors. When a representation is computer generated, can be comprised of one or more pixels (in 2D) or voxels (in 3D). In the present document, while the terms pixels and voxels can be used separately for 2D and 3D, respectively, it will be understood that the term “voxel” can includes the term and definition of “pixel”.

The following is a detailed description of embodiments of the disclosure depicted in the accompanying drawings. The embodiments are in such detail as to clearly communicate the disclosure without limiting the anticipated variations of the possible embodiments and may encompass all modifications, equivalents, combinations and alternatives falling within the spirit and scope of the present disclosure. It will be appreciated by those skilled in the art that well-known methods, procedures, physical processes and components may not have been described in detail in the following so as not to obscure the specific details of the disclosed invention.

While the present disclosure may seem to mainly focus on radiography and computed tomography (subsets of X-ray imaging), it will be appreciated by someone skilled in the art that the method and apparatuses proposed herein may be used or modified to be used for or in combination with any alternative compatible X-ray imaging technique and/or device. In the present disclosure, the term computed tomography (CT) can comprise medical or industrial (e.g., quality control or airport security, etc.) radiography, X-ray imaging, computed tomography scans as traditionally defined in the field, protectional radiography or a combination thereof.

The present disclosure presents a novel method and an apparatus for analyzing a measurement of incident X photons of computed tomography that can utilize diffused/scattered X photons to characterize some properties of the scanned object. In an embodiment, the measured scattered X photons can be used to generate scattering images, to determine the scatter properties (e.g., the scattering coefficient) in a scanned material, which can be used to determine the nature or alternative characteristics of the material. For example, the proposed method may give better diagnostics by quantifying various characteristics of the materials such as the quantity of iron in a tumor and its degree of vascularization, which may confidently, easily, more efficiently and/or more rapidly identify the nature of the tumor (e.g., cancerous or not), for example.

An absorption image, generally used to characterize the absorption coefficients and/or the density of the various structures of the scanned region of the object, can therefore be improved by incorporating data from the scattered photons such that it can provide additional/complementary information about these structures comprised in the absorption image, which may be information about the possible nature or composition of these structures.

In the prior art, two separate scans were required to have an absorption and a scattering image. It will be appreciated that the scattered photons utilized by the proposed method herein were previously regarded as noise or signal pollution in absorption X-ray imaging. However, the method and/or apparatus proposed herein can be combined with the method and/or apparatus described in the PCT publication WO2020093140 in order to generate (e.g., simultaneously) both the absorption and scattering images from the same X-ray pulse at a normal X-ray dose and even at a reduced X-ray dose.

The method approximates possible two-dimensional (2D) or three-dimensional (3D) locations of scattering events of X photons for each acquisition by analyzing the measurement of the elapsed time from emission to detection of photons (time-of-flight of detected photons). Various types of X-ray imaging systems and setups (e.g., computed tomography systems and setups) can be used to utilize the proposed method. Since the method is based on time-of-flight measurements, the method can be used with an apparatus comprising a source for producing and emitting a short burst of photons (e.g., short X-ray pulse) on one side of the object to scan, a time-sensitive photon detector for identifying the location of detection (detection location) and the time at which each incident photons are detected and a computing apparatus, which may be a processor (CPU, GPU, FPGA, etc.) or image processor. With this configuration, the time-of-flight may correspond to the elapsed time measured from the time the photon is emitted from the source to the time of detection of the photon by the time-sensitive detector. In some embodiments, these times of emission and of detection may be included in and provided by time-dependent X-photon detection data, which may be used by a processor to determine the various time-of-flights. In some embodiments, the time-dependent X-photon detection data can alternatively or additionally include and directly provide the time-of-flights.

In the context of the present disclosure, it will be understood that the term “time-of-flight” may be, in some specific cases and steps (e.g., when detecting/measuring/receiving the time-of-flights, when building the TPSF, or when identifying or selecting the scattered photons scattered once from the ballistic photons and the scattered photons scattered more than once), generalized to and/or substituted by a time/timestamp (e.g., an elapsed time or time of detection) that may essentially comprise the time-of-flight or which can be used to determine/calculate the actual time-of-flight. It will be appreciated that the concept, definition or value of a time-of-flight can encompass, be transposed to or translated into equivalent time values (e.g., absolute or relative times, time of detection, timestamp). Therefore, through the current disclosure the term time-of-flight may not be limited to the strict definition of a specific value of elapsed time between the emission of a X-photon at the source and its detection at the detector. For example, the time-of-flight can encompass a relative time of detection (e.g., a time relative to or elapsed from an activation of the detector), the concept being that, although the monitored or known information may not be detailed enough to do so (e.g., to avoid/reduce some measurement or time resolution requirements), the considered value of time could theoretically be used to determine or calculate the true time-of-flight (i.e., the value of elapsed time between the emission and detection) if additional information was available (e.g., the time between the releasing of the source pulse and the activation of the detector). For example, a detection time may be converted to a time-of-flight using a lookup table or known conversion factor or added correction value.

The time width of a burst of photons can be defined by its average full width at half maximum (FWHM). A short pulse or burst of photons can be defined here as being of time width of preferably less than about 100 picoseconds. Furthermore, the rising edge of the X-ray pulse can be less than about 300 picoseconds and is preferably of less than about 150 picoseconds.

### Selection of the Scattered Photons

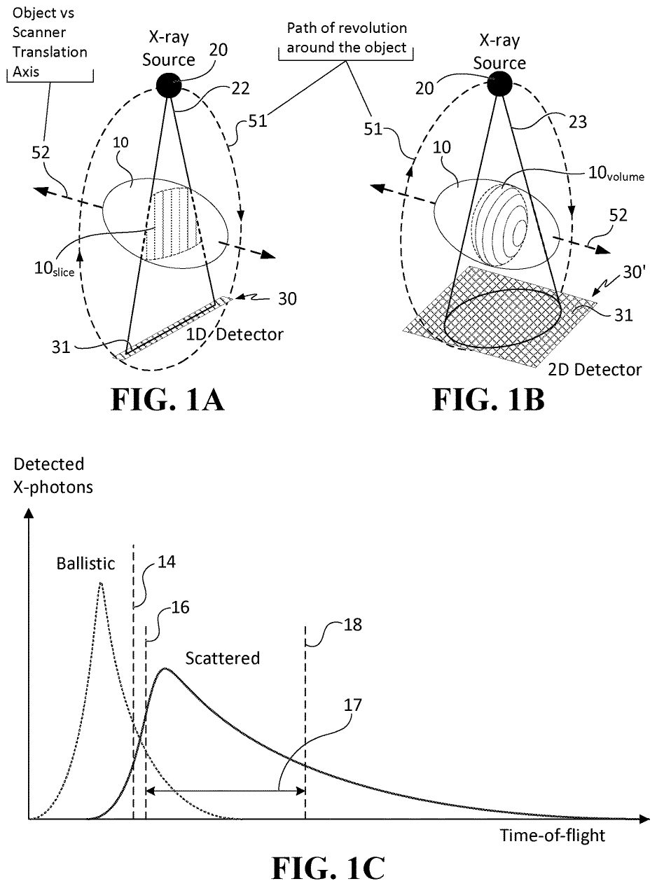

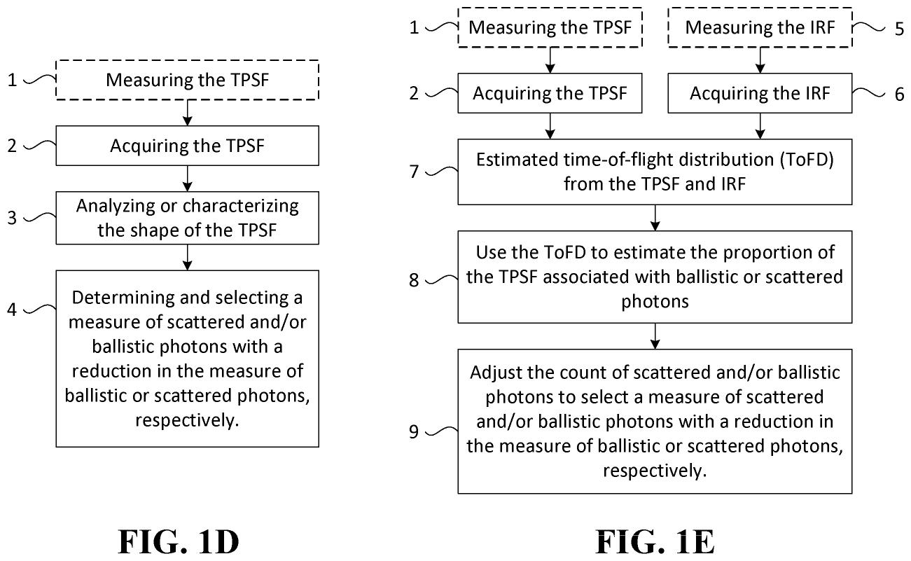

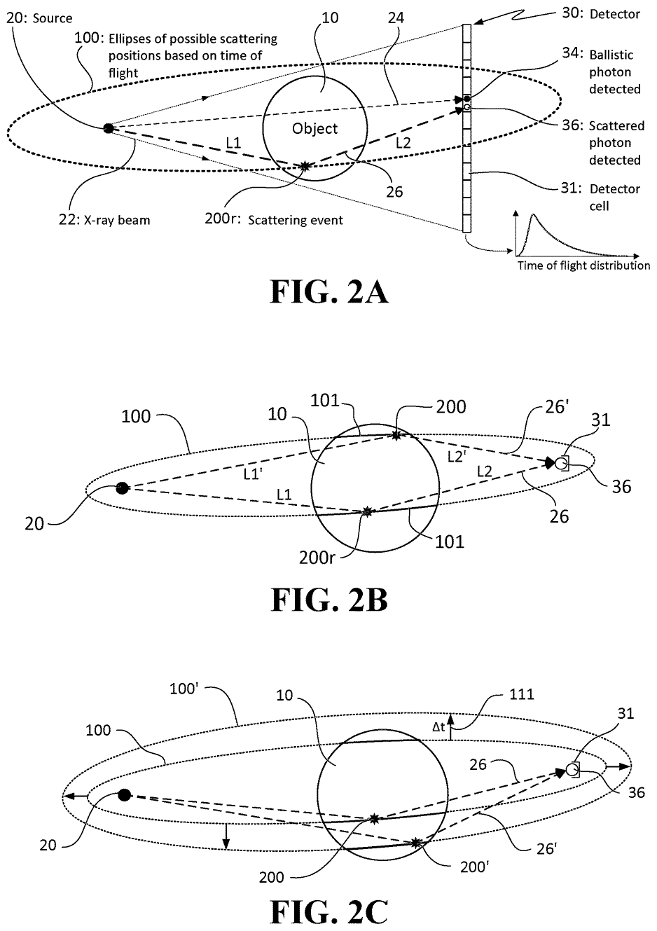

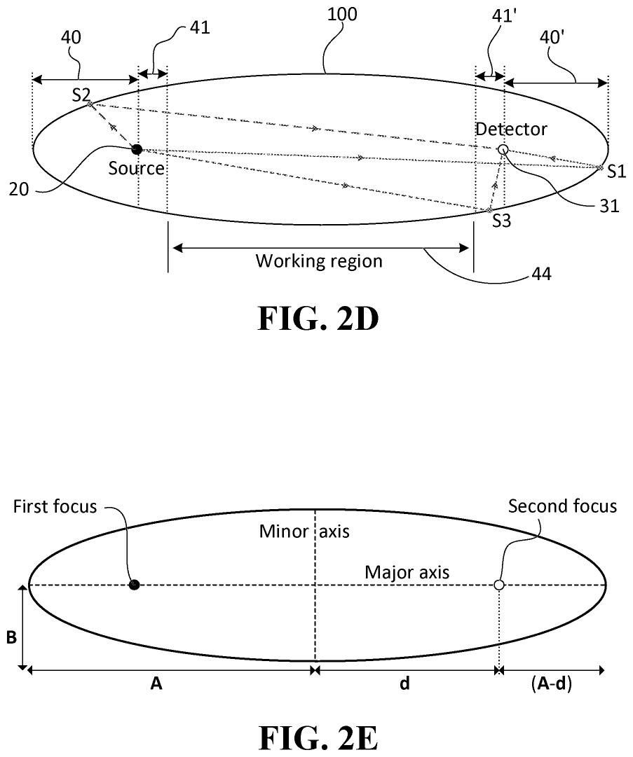

Note that the source can spatially emit photons within various angular apertures (e.g., in a conical aperture, a fan-beam aperture, a thin cylindrical aperture (collimated beam)), and the pulse/beam can have any temporal shape (e.g., square, rectangular, or triangular, or any 2D shape), can be diverging (e.g., where the pulse is spatially broader on the detector side than at the source, as illustrated in FIGS. 1A, 1B and 2A), parallel (e.g., where the size of the pulse is the same at the source and at the detector) or converging (e.g., where the size of the beam is the broader at the source than at the detector).

In some embodiments, the proposed method can be improved by using systems that comprise a time-sensitive detector that can have a timing resolution precise enough to allow the processing of data in order to effectively remove the impulse response (spreading over time) of the instruments/components of the scanner (e.g., of more than about 10 picoseconds to about 300 picoseconds). It will be appreciated that, in some embodiments, this may be enough to discriminate ballistic photons from non-ballistic photons (e.g., scattered photons) with a fairly satisfactory level of precision.

Scattered photons are defined herein as including photons that were scattered by Compton scattering and/or by Rayleigh scattering.

As illustrated in the perspective view of a schematic drawings of a scanner in FIG. 1A, some embodiments of the X-ray imaging device can comprise a pulsed X-ray source 20 that can generate a short photon pulse having a 1D wavefront that traverses a 2D area (e.g., fan-shaped beam 22) directed at a time-sensitive detector 30 comprising a one-dimensional array of X photon photodetector 31 that can detect incident photons (ballistic and scattered) and measure their time-of-flight. The embodiment of FIG. 1A can be used to acquire a 2D slice 10_{slice} of an object 10 (here illustrated as an ellipsoid shape) of interest and, in some embodiments, can do so from various angular orientations around the scanned object 10. In some embodiments, the source 20 and the detector 30 can simultaneously revolve around the object 10 at various (e.g., incremental) positions along the revolution/orbital path 51 which is illustrated as a double-lined circle taking an oval shape due to the perspective view and can be in the same plane as the fan-shaped beam 22. It will be appreciated by someone skilled in the art that a three-dimensional characterization of a volume of interest may be completed by considering and merging a plurality of 2D measurements (e.g., sub-acquisition) taken at various angular position around the object 10. This can be used to calculate and generate 2D or 3D images comprising calculated pixels (2D image unit) or volume pixel (3D image unit), i.e., voxel, respectively.

In an embodiment, the X-ray imaging device (e.g., a radiographic imaging device or a computed tomography scanner) can be designed to change its position relative to the scanned object 10 and can, for example, be translated along an axial translation axis 52 (illustrated as dashed arrows perpendicular to the revolution path 51) around which the device can rotate along a revolution path 51 to revolve around the scanned object 10. Alternatively, the object (e.g., patient) can be translated along an axial translation axis 52 relatively to the source and detector. It will be appreciated that in some embodiments the X-ray imaging device can be used to create a 2D radiography image if the source and detector pair is not rotating around the object or a 3D computed tomography image if the source and detector pair is rotating around the object (along the revolution path 51) and provide enough projections to create a computed tomography image for each translation position along the translation axis 52.

In some embodiments, the 2D or 3D image results of the proposed method can be generated by considering or combining (e.g., with an algorithm, an image processor, a CT image processor or others) a collection of various characterizations of the readings/measurements, each associated to and resulting from a sub-acquisition (i.e., each time the source emits a pulse of photons at a fixed position) that may be repeated at a same position or can be completed at various positions of the object relative to the source and detector (e.g., various/incremental revolution/orbit positions of the source and detector, and/or various/incremental axial positions of the object). Various embodiments of a radiographic imaging device may be used to generate for each sub-acquisition a one-dimensional (1D) or a two-dimensional (2D) characterization of the possible position of the scattering events.

As illustrated in FIG. 1B, some embodiments of the X-ray imaging device can comprise a pulsed X-ray source 20 that can generate a short photon pulse having a 2D wavefront (e.g., circular wavefront expanding inside a cone) that traverses a 3D area (e.g., cone-shaped beam 23) directed at a time-sensitive detector 30′ comprising a two-dimensional array of X photon photodetectors 31 that can detect incident photons and measure their time-of-flights. The embodiment of FIG. 1B can be used to complete a scan by acquiring a sequence of radiographic images when the source and detector couple are not rotating around the subject or a 3D slice 10ᵥₒₗᵤₘₑ of a scanned object 10 of interest and can do so from various angular orientations around the object 10 (e.g., along the revolution path 51). In some embodiments, a 3D X-ray beam can take the shape of a thin rectangular base pyramid that can resemble a 3D embodiment of the fan-shaped beam and the detector can be a 2D detector of the dimension of the base of the beam. In some cases, the fan-shaped beam and the thin elongated 2D detector (e.g., having a dimension of about 1 to 20 cm by about 140 to 200 cm) can be used to complete scans/acquisitions having a reasonable number/count of detected scattered photons to generate a scattering image.

It will be appreciated by someone skilled in the art that the combination of the geometric characteristics of the “travel path” of the photon pulse (e.g., 1D pen-shaped, 2D fan-shaped 22 or 3D cone-shaped 23) can dictate how many dimensions a single sub-acquisition can characterize. For example, a 1D pen-shaped pulse of X photons (i.e., pulsed X-ray beam) can be used, with a 1D detector 30 or 2D detector 30′, to determine the position of scattering of scattered photons along the path of the one-dimensional pulse. Alternatively and for example, a 2D fan-shaped pulse of X photons can be used, with a 1D detector 30 or 2D detector 30′, to determine the position of scattering of scattered photons within the path of the two-dimensional pulse 22 (i.e., in the fan-shaped area traversed by the photons) illustrated in FIG. 1A. Alternatively and for example, a 3D coned-shaped pulse of X photons can be used, with a 1D detector 30 or 2D detector 30′, to determine the position of scattering of scattered photons within the path of the three-dimensional pulse 23 (i.e., in the cone-shaped volume traversed by the photons) illustrated in FIG. 1B.

It will also be appreciated that, in some embodiments, the dimension of the detector (1D or 2D array of detector cells) can influence the accuracy of the characterization or can reduce the needed dose of photons emitted by the source (e.g., X-ray dose) to achieve a same level of accuracy, since the 2D detector can detect more scattered photons than a 1D detector, and can therefore increase the number of counts of detected scattered photons, which can lead to an improved accuracy of the characterization result and of the resulting image(s).

The embodiments described herein can allow the usage of cone-beam computed tomography with even larger volumes as illustrated in FIG. 1B. Cone-beam computed tomography can have an advantage over the standard fan beam computed tomography helicoidal geometry (i.e., a combination of revolution 51 and translation 52 motions so that the source and detector move along a relatively helicoidal path around the object) mostly used nowadays since it does not require linear translation of the patient. The simplicity of the mechanical parts of cone-beam computed tomography reduces the form factor of the scanner that may be needed to use such a system directly in an operating theater, for example.

It will be appreciated that the proposed method may yield better results using state-of-the-art X-ray sources that can have an X-ray beam pulse having a shorter spread of emitted photons in time (e.g., ultra-short X-ray pulses of less than about 150 picoseconds full-width at half maximum—FWHM) and/or state-of-the-art detectors (e.g., a silicon photomultiplier or all digital derivatives thereof).

In the prior PCT publication WO2020093140, the detected photons are filtered or discriminated using their associated measured time-of-flight, where the photons detected before a chosen temporal/time-of-flight threshold (e.g., scatter threshold 16) and/or cut-off (e.g., ballistic cut-off 14 and/or scatter cut-off 18), as illustrated in FIG. 1C, can be considered as ballistic photons that may not have interacted with the scanned object to better reconstruct the resulting absorption image. In some embodiments, the absorption image can be generated from a number of counts of detected ballistic photons for various positions at the detector (e.g., a count of ballistic photons for each detector cells/pixels of the detector). For example, each count can contribute to the absorption value and each position of the detector cells can be associated with the position of a pixel of the absorption image.

The temporal ballistic cut-off 14 may be a time-of-flight value (timestamp) serving as a discrimination threshold that can be used to identify the photons detected before (having a time-of-flight smaller/lower than) the time-of-flight value associated with the ballistic cut-off 14 as being ballistic photons and to identify the photons detected after and having a time-of-flight greater/above/higher than the associated time-of-flight value as being scattered photons (non-ballistic-photons).

In some embodiments of the method proposed herein, X photons that have been scattered more than once may be associated with a time-of-flight above the temporal “scattering cut-off” 18. The temporal scattering cut-off 18 may be corresponding to an approximative value associated to the time-of-flight where a significant number/count of scattered photons that have been scattered more than once start to be detected. Therefore, in some embodiments, the X photons having a time-of-flight above this scattering cut-off 18 may be discarded (not considered) for obtaining an image. The use (considering) of X photons having a time-of-flight above scattering cut-off 18 may lead to adverse effect on the accuracy of the results since it may not be possible to accurately approximate/calculate the possible scattering positions of multiple consecutive scattering events of a single X photon solely based on its time-of-flight and detection location.

In embodiments where only the scattered photons are detected, the time-sensitive X-ray detector may only be activated after a set time-delay threshold (e.g., after a time-delay or time-of-flight associated with ballistic photons) or within a set time-range (e.g., a time-range or time-of-flight range associated with photons scattered once), between a time-delay threshold 16 and a cut-off time 18, and may therefore only detect photons after a set a time-of-flight threshold 16 or within a time-of-flight range 17, wherein all photons detected by the time-sensitive X-ray detector are considered to be scattered photons. In some embodiments, all of the incident X photons (ballistic and scattered) can be detected, and the photons detected before a time-of-flight threshold 16 or outside a time-of-flight range 17 can be filtered out, discarded or put aside. The remaining photons can be considered photons scattered once and can be used in the proposed method.

The method proposed herein can use at least a portion of these non-ballistic photons to approximate a measure of scattering properties of various parts (sub-regions/volumes) of a scanned object and to generate a scattering image, which can be used separately or complementarily to absorption images to better identify the nature of these parts since absorption and scatter parameters may be different for materials with similar densities as described herein below.

Most of the non-ballistic photons may be scattered photons, which are photons that interacted with an object or matter without being absorbed and are instead scattered as dictated by the Compton scattering law. It will be appreciated that, as illustrated in FIG. 2A, a scattered photon 36 takes more time to go from the point of origin (e.g., the source 20) to a given destination (e.g., the detector 30) than it takes for a ballistic photon 34 (a photon with straight travel path between the origin to the same destination) since they both have the same speed but the ballistic photon has less distance to travel. This can lead to a noticeable disparity between the average time-of-flight of the ballistic photons and the average time-of-flight of the scattered photons from a single pulsed beam as schematically illustrated in FIG. 1C. FIG. 1C shows an exemplary distribution of detected ballistic photons and scattered photons as a function of the time-of-flight (e.g., TPSF) as well as exemplary drawings of various filtering or selection limits such as: a ballistic cut-off 14 as introduced in PCT publication WO2020093140, a “scattering threshold” 16 that can be used to designate at which the detected photons can be considered to be scattered photons, and an optional “scattering cut-off” 18 that can be used to designate a limit at which the detected photons are considered to have been scattered twice. In some embodiments, the scattering threshold 16 can have the same value as the ballistic cut-off 14, but should not be smaller.

In some embodiments, a measure of the time-of-flight of the photons can be used to form a histogram of the time-of-flight (e.g., a number of detected photons for various intervals of time-of-flight) called the temporal point-spread function (TPSF). The resulting dataset of the TPSF can then be used to better estimate the number of ballistic and/or scattered photons detected. This estimation may be utilizing the TSPF of the instruments-data of control measurements without any object between the source and the detector.

Therefore, it is understood that generating or using the TPSF is only one of the specific case of using time-of-flights.

An alternative approach, so-called the TPSF approach, may be used to move away from a standard time-of-flight X-photon discrimination on a photon-by-photon basis to remove ballistic photons from the measurement of scattered photons. It was previously discovered (for details, see PCT publication WO2024/092370 published 10 May 2024, which is incorporated herein by reference) that measuring the elapsed time from emission to detection of X-photons (time-of-flight) distribution for all X-photons for each detection location (i.e., detector cell 31), also known as a temporal point spread function (TPSF), can further be used to relax the temporal resolution requirement at the detector. This approach involves the acquisition of a statistically valid population of detected X-photon to form a TPSF dataset, however, this can be achieved efficiently over a large number of X-ray source pulses. This approach can tolerate a greater time variability in an X-ray source's pulses and in an X-ray detector's time resolution while still providing effective elimination of the measurement of ballistic X-photons to better discriminate/select the scattered photons.

In an embodiment, this can comprise: measuring the Instrument Response Function (IRF) for each detector pixel with an acquisition without any object between the source and the detector; scanning a subject-of-interest to measure its corresponding TPSF of photon impinging on each pixel; generating a TPSF dataset; correct the TPSF dataset with the IRF dataset using a temporal deconvolution to yield the estimated time-of-flight distribution; and estimate the proportion of ballistic and/or scattered photons from the estimated time-of-flight distribution to correct the counts per detector.

Since it may be difficult to accurately identify scattered photons in systems with large IRF, the proposed invention can use the TPSF directly to estimate the amount of detected scattered and/or ballistic photons regardless of which exact photon has scattered or not. This can be notably done by first measuring the IRF, and then computing the deconvolution of the TPSF by the IRF to provide an estimated time-of-flight distribution (EToFD). The IRF can either be measured or estimated. To measure the IRF, an acquisition with nothing between the source and the detector may be completed. The IRF can correspond to the output of the system for a delta function. Since ballistic photons may be the only detected photons, which can have a time-of-flight distribution that can act like a delta function, the measured distribution can be considered as the IRF. The IRF can also be estimated based on the expected pulse width and expected random jitters from the detectors. In some embodiments, this approach may result in a less accurate IRF estimation.

It will be appreciated that, for some embodiments, the exact shape of the time-of-flight distribution may not be crucial, since the ratio between the initial peak of ballistic photons and the tail of scattered photons can be the most important characteristic when it comes to estimating the contribution of the non-ballistic photons (e.g., scattered photons) to the count of ballistic photons considered to generate the absorption image. In fact, when it comes to discriminate between ballistic and scattered photons, an accurate estimate of the time-of-flight distribution may be sufficient. Therefore, simpler algorithms may be used.

In some embodiments, the noise of the measured TPSF can then be removed since the deconvolution can be an ill-posed problem. This can notably be done by first doing an Anscombe transformation on the TPSF, completing a wavelet denoising and finally by determining an inverse Anscombe transformation.

In some embodiments, the same denoising methodology can also be applied to a measured IRF. The deconvolution of the TPSF dataset by the IRF dataset can be computed using several methods. One such method is by minimizing the following function:

$${‖ Ax - b ‖}_{2}^{2} ,$$

where, A can be a lower triangular matrix whose elements are the IRF values, x can be a vector containing the current estimation for the time-of-flight distribution (ToFD) and b can be a vector containing the TPSF data. The initial guess, which may highly impact the output of the minimization, can assume that every photon is ballistic and may therefore assume that the ToFD is a delta function multiplied by the sum of the TPSF. This yield an estimated ToFD further called ETOFD.

Once the EToFD is computed, it can be used to estimate the number (counts) of detected scattered photons and/or ballistic photons. In some embodiments, this can be achieved by first identifying the maximum of the EToFD which can correspond to the ballistic contribution before integrating it. The integration bounds may be selected to include the range of values that form the “peak” of the distribution. For example, it may include, in one embodiment, the values contiguous to the maximum that are larger than a tenth of the maximum of the EToFD. This is analogous to applying a discrimination threshold (e.g., a scattering threshold 16 or a ballistic cut-off 14 as illustrated in FIG. 1C) to a more precise version of the TPSF, which can instead consider the proportion of the total count of detected photons instead of a predetermined time-of-flight serving as this discrimination threshold. Neural networks, also known as artificial intelligence, are known as a good estimation tool. The neural network may be programmed into a processor or any computing device that may be part of, or integrated into, or coupled with or into a separate device connected to the X-ray apparatus. In some embodiments, the neural network can be trained to use the shape (at least one shape parameter) of the TPSF in order to estimate the number of scattered and/or ballistic photons considered to generate the X-ray image.

It will be appreciated that these are only some of the possible methods that can be used to estimate the count of scattered and/or ballistic photons. In some embodiments, part of the distribution may be summed (integrated) or the value of the peak may be used, for example. The method to be used may be chosen as a function of how the ETOD is generated.

FIG. 1D presents a block diagram of the steps of a possible embodiment of the proposed method. A first step 1 may be required to complete an X-ray scan and acquire the time-of-flight measurements of photons detected by an embodiment of a computed tomography, for example. Step 2 may be a following step if the TPSF is generated from the measurement data or can, in some embodiments, be a first step comprising acquiring a TPSF of a previously completed measurement, which may be extracted from some electronic system's memory. The following step 3 can comprise analyzing the distribution (shape) of the TPSF to characterize it. It will be appreciated that, throughout the present disclosure, the shape of the TPSF relates to the collection of all characteristics of the distribution of the histogram of the time-of-flight of the detected photons and it is understood that these characteristics or collection of all characteristics (i.e., the shape) of the TPSF may be comprised (explicitly, implicitly, vaguely, cryptically, etc.) in the associated data, namely the TPSF dataset, which may be acquired with the detector of the imaging apparatus of extracted from a database or memory. In an embodiment, the following step 4 can comprise using the characteristics of a characterized TPSF to select the portion of the count of detected photons that should correspond to the scattered and/or ballistic photons (e.g., discarding the portion that should correspond to non-ballistic photons) to determine a measurement of scattered and/or ballistic photons with a reduction of ballistic or scattered photons, respectively. For example, the identified characteristics of the shape of the TPSF may be used to find, within a lookup table (e.g., associated with a given imaging apparatus), the proportion to be considered.

The X-ray image can be generated using a CPU, a GPU, an FPGA or a combination thereof. It will be appreciated that the X-ray image can be generated using state of the art or conventional components of imaging apparatus of the current prior art or to be developed. The imaging can be performed with a computed tomography imaging apparatus able to make time correlated single photon counting. For X-ray radiography the image can normally be obtained directly from the counts of photons per detector pixel. The image generator function as normally observed in computed tomography, where the image can be reconstructed using any conventional reconstruction algorithm such as the filtered-back-projection (FBP) or the Feldkamp-Davis-Kress algorithm for cone-beam CT (FDK) or any analogous algorithm and programs. Such algorithms can be implemented as part of the reconstruction toolkit (RTK) and the astra-toolbox libraries. In some embodiments, the estimated count of ballistic photons can preferably be normalized to the number of emitted photons.

FIG. 1E presents a block diagram of the steps of a possible embodiment of the proposed method comprising some steps that are similar to the steps of the block diagram of FIG. 1D. This can include step 1 for measuring the TPSF's data, and a step 2 to acquire or generate the TPSF. This embodiment can further comprise a step 5 for measuring the temporal resolution and uncertainties of the X-ray imaging apparatus, which may be used to generate, in step 6. In some embodiment, step 6 can comprise retrieving or acquiring the IRF of a previously completed measurement, which may be extracted from some electronic system's memory. In a following step 7, the TPSF can be temporally deconvolved by the temporal resolution function (e.g., IRF) to estimate a more accurate distribution of the detected photons (e.g., time-of-flight distribution). Step 8 can comprise using and/or processing the estimated ToFD (EToFD) to estimate a portion of these distributions associated with ballistic photons. In a following step 9, this can be used to adjust the considered measure (count) of ballistic photons with a reduction of scattered photons. This adjusted count can then be used for the other steps of the proposed method as it will be described in the following.

### Data Interpretation and Scattering Representation

It can be estimated that the number of scattering events experienced by a photon in a material depends on the electron density distribution (ρ) divided by the energy of the incident photon (E), while its absorption depends on the cube of the atomic number of the attenuating medium (Z³) divided by the cube of the energy of the incident photon (E³). As a function of energy, the quantity of scattered photons can be significantly more than that of ballistic photons. As an example, the ratio of the number of scattered photons to that of primary (ballistic or non-deflected) photons, so-called the scatter-to-primary ratio (SPR) can exceed 300% for a phantom of water having a thickness of about 200 mm. However, photons that were scattered once represent only 13% of all scattered photons.

It will be appreciated that, in some embodiments, an additional time sensitive limit can be considered to filter out or discard scattered photons that were detected after a chosen time-of-flight which may be considered to correspond to photons that were scattered more than once (multiple scattering).

FIG. 2A illustrates a schematic drawing a time-of-flight acquisition for a ballistic 34 and a scattered 36 detected X photon with a 2D computed tomography scanner comprising a pulsed X-ray source 20 that can generate a 2D fan-shaped beam 22 that can send a pulse of X photons at an object 10 and towards a detector 30. In this embodiment, the detector 30 is a one-dimensional detector array of detector cells 31 (a line of detector cells) similar to the one illustrated in FIG. 1A. A detector cell 31 can be a subsection of the detector array 30, which can have a small surface area and can be associated with a given position (detection location) within the detector array 30. The detector cell 31 may be considered to be a “pixel” of the detector array 30. Here a 2D beam and a 1D detector are considered to simplify the description of the fundamental principles and dynamics on which the proposed method is based. This schematic drawing further shows two types of incident X photons that may be considered by the proposed method and apparatus. The Figure shows a detected ballistic photon 34 having a direct travel path 24 that is typical if not absorbed nor scattered, and a detected scattered photon 36 having a deviated (deflected) travel path 26 compared to a straight line. In this example, both photons originating from the source 20 (origin) at the same time, are detected at the same position (destination) on the detector array by the same detector cell but at different times (i.e., both having different times-of-flight). This specific case will help to appreciate that, since both ballistic and scattered photons have the same speed, the total length of the deviated travel path 26 must be longer than the direct travel path 24. This deviated travel path is even longer when the photon is multiply scattered in the object.

For a fixed origin and destination, a given time-of-flight of a photon that has been scattered only once may only correspond to a specific total length (L) of the associated full deviated travel path since, for photons, the traveled distance is given by L=c× ToF, where c is the speed of light in a vacuum. One can appreciate that the refractive index is, to a good approximation, equal to 1 for photons in the X-ray energy range and thus the speed of X photons is effectively equal to the speed of light in vacuum.

In the case of a 2D scanner of FIG. 2A, the propagation of a detected ballistic or singly scattered photon lies in a plane because the photons are emitted in a plane and detected in that same plane. As illustrated, a photon that experiences a single scattering event travels along two straight line segments, the first segment L1 from the source 20 to the scattering event 200r and the second segment L2 from the scattering event 200r to the detector cell 31. The total time-of-flight of that photon, which can be measured and may be known, is the sum of the time-of-flight along the first segment (ToF1) and the time-of-flight along the second segment (ToF2). Thus, knowing the total time-of-flight (ToF=ToF1+ToF2) of the photon can allow knowing the total length (L) traveled by the scattered photon 36 that is the sum of the length of the two segments (L=L1+L2=c×ToF1+c×ToF2=c×(ToF1+ToF2)=c×ToF). These two lengths need not to be known individually, since the location of the scattering event is not known, but as just seen, the sum of these two lengths is known. By definition, an ellipse is a locus of points for which the sum of the lengths of two chords (line segments) is constant. Hence, the geometrical locus of points on which the position of the single scattering event 200r of the measured scattered photon 36 can possibly lie is an ellipse having as its foci the X-ray source 20 and the detector cell 31 at which it is detected. FIG. 2B shows an ellipse of possible scattering positions 100 for a detected scattered photon having a given time-of-flight between the X-ray source 20 and the detector cell 31. Knowing the shape and the position in space of the object, it is possible in principle to restrict the full ellipse of possible scattering positions/locations 100 (illustrated as the dotted ellipse) to two sub-arcs 101 (illustrated as the two full arcs within the object 10) of the ellipse corresponding to possible scattering positions/locations 200 (neglecting the scattering of air, which is indeed negligible). Thus, for a detected scattered photon 36, it is not possible to determine the exact position where the single scattering event occurred 200r, but a restricted locus of possible positions is known. In the case of a 3D scanner, the same considerations apply, with the difference that the ellipse becomes an ellipsoid and ellipse sub-arcs become ellipsoid surface sub-areas, with the X-ray source and detector cell being at the ellipsoid foci.

FIG. 2C shows the relationship between the measured time-of-flight (e.g., longer measured time-of-flight versus a shorter one) and the ellipse of possible positions of a scattering event 100. For this example, two scattered photons having been scattered once at different positions 200/200′, having different time-of-flights and being detected at a same position on the detector (by the same detector cell 31). A shorter time-of-flight is associated with a shorter possible total length of travel path 26 and a longer time-of-flight is associated with a longer possible length of the travel path 26′. Therefore, the ellipse of possible scattering locations 100 for the photon with a shorter time-of-flight is more eccentric than the ellipse of possible scattering positions 100′ for the photon with a longer time-of-flight. In other words, the eccentricity of the ellipse can increase with the time-of-flight of the detected scattered photon.

Similarly, noise on timing measurement may add uncertainty on the ellipse's thickness and can be represented as a thickened elliptical path (in this case 100 and 100′ would be thick arcs). This noise can be considered in the image reconstruction process.

In practice, it may be that the exact position and shape of the object are unknown (although a standard X-ray CT image can provide this information). In such a case, instead of considering the possible scattering locations inside the object, a “working region” that designates a space between the source 20 and the detector cell 31 can be considered to reduce the extent of the possible arcs of possible scattering locations considered. FIG. 2D illustrates such a working region. This Figure helps appreciate that a measured scattered photon cannot possibly have been scattered only once on the ellipse portions in the region behind the directional X-ray source (e.g., at position S2), corresponding in this embodiment to the region 40 on the left side of the source 20, since the beam may only be directed in the direction of the detector array (e.g., towards the right side of the source). The measured scattered photon cannot possibly have been scattered only once on the ellipse portions in the region behind the detector array (e.g., at position S1), corresponding in this embodiment to the region 40′ on the right side of the detector cell 31, since the detector array faces the source 20 in order to detect its beams (e.g., towards the left side of the detector cell 31). FIG. 2D further introduces buffer zones 41 and 41′ which may be considered for embodiments where the subject may be required to be placed at a certain distance from the source 20 and/or the detector 30. So, in some embodiments, the scattering locations S1, S2 and S3 may not be proposed as possible scattering positions since they are not within the working region 44. It will be appreciated that the known shape of the X-ray beam (e.g., fan-shaped or conical) may further be considered to limit or define the working zone, which can then be comprised within the beam's shape.

As mentioned previously, the possible locations of the scattering event for a detected scattered photon can be an ellipse as illustrated in FIG. 2E when it is scattered once before reaching the detector. In appropriate x and y coordinate axes, the equation of the ellipse can be written as

$$\frac{{x}^{2}}{{A}^{2}} + \frac{{y}^{2}}{{B}^{2}} = 1$$

and d=\sqrt{A²−B²}, where A is the length of the semi-major axis on which the foci (first focus and second focus) are placed, B is the length of the semi-minor axis which is perpendicular to the semi-major axis and dis the length from the center of the ellipse to either of the foci. For each individually measured scattered photon, the source and the detector are the foci of the ellipse. The location of the scattering event is unknown, but one of the potential trajectories of the photon allows the computation of A. Ignoring the actual location of the subject which would cause the scattering event, the photon could go from the source in the direction of the detector, go through it, scatter behind it and return to the detector where it is absorbed. This trajectory is described with L=(2d)+2 (A−d)→A=L/2. Next, d may be computed from the known distance between the source and the detector cell of the detector measuring the photon divided by 2. It will be appreciated that d may also be calculated from a time-of-flight of a corresponding ballistic photon detected by a corresponding detector cell since 2d=c×ToF_{ballistic}. Also, B may be calculated from A and d, since B=\sqrt{A²−d²}. The equation of the ellipse can therefore be known to determine various x-y pairs to generate corresponding curves of possible scattering locations using the values A and B calculated using the photon's time-of-flight and the distance source/detector (2d). A similar reasoning can be used to define the values for the ellipsoid equation:

$$\frac{{x}^{2}}{{A}^{2}} + \frac{{y}^{2}}{{B}^{2}} + \frac{{z}^{2}}{{C}^{2}} = 1$$

### in the 3D Case.

The proposed method can be used or performed by an apparatus in order to generate at least one of a scattering representation, an absorption representation, or a combination thereof. In the present disclosure, it will be understood that a representation is not limited to a visual representation and can include data that can be used (e.g., later be used) to generate a visual representation. For example, a representation may be at least one data set that can comprise information (e.g., scattering count, scattering location, values and/or coefficients) associated with special coordinates (e.g., pixel and voxel matrices).

The scattering representation can be determined from or based on a plurality of possible locations of single scattering events that may be determined using corresponding time-of-flights and detection locations.

The absorption representation can be determined from or based on a plurality of detection locations of detected ballistic photons using detection locations and, in some embodiments, the corresponding time-of-flight.

In some embodiments, the scattering representation can be determined from or corrected/refined using the detected locations of the ballistic photons (e.g., absorption representation).

An image may be reconstructed based on scattered photons. The proposed method can comprise a reconstruction method that can correctly identify possible scattering locations on ellipses. For the sake of simplicity, the present disclosure focuses on the 2D case, but ellipsoids can be considered in the 3D case. The proposed method can sum, count or superimpose the number of scatterings in various materials, which can be used to approximate their electron density. The proposed method can reconstruct various representations of the scanned object including its various subparts composed of various materials.

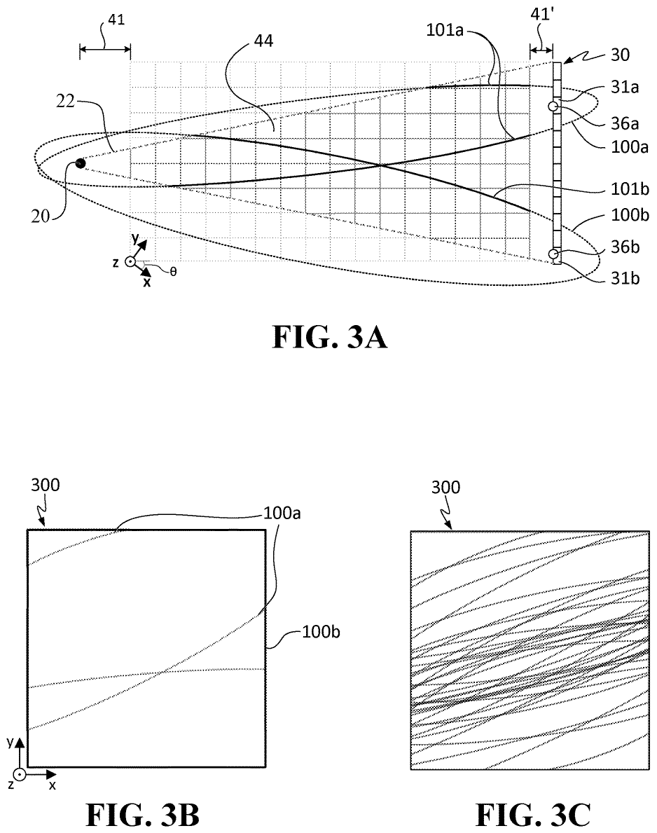

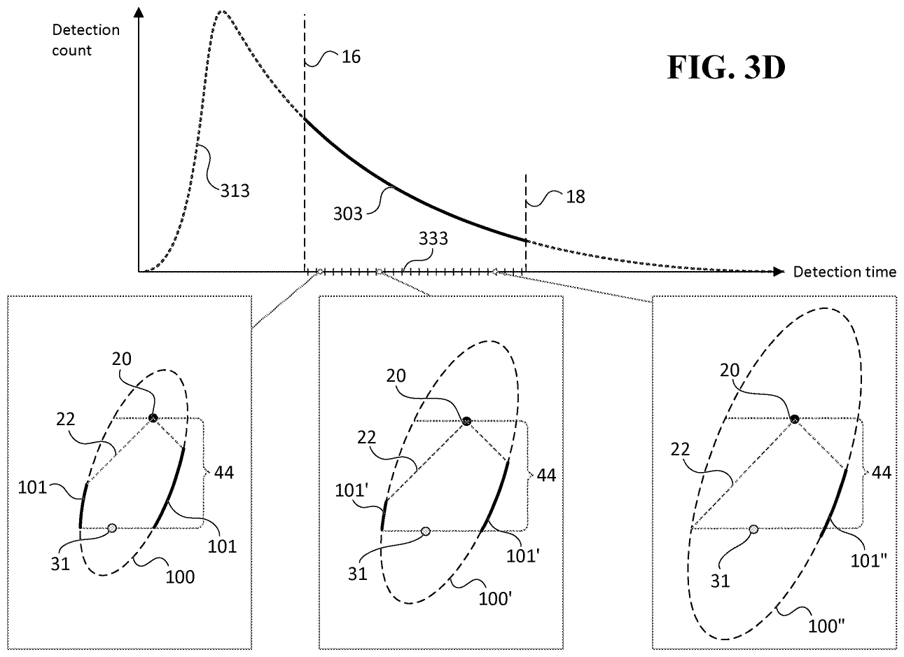

In some embodiments, the reconstruction method can include defining the working region 44 of the scanner setup, which can be geometrically calculated using the dimensions of the detector array 30, the shape of the X-ray beam 22, the distance between the X-ray source 20 and the detector 30, and/or any buffer zone 41. FIG. 3A illustrates an exemplary 2D acquisition in which two scattered photons 36a/36b have been detected. The reconstruction method can include determining the arcs of possible scattering locations 101a/101b within the predetermined working region 44 of the calculated ellipses of possible scattering positions 100a/100b of each of the detected scattered photons 36a/36b using the time-of-flight and the distance between the source 20 and the corresponding detector cell 31a/31b.

It will be appreciated by someone skilled in the art that a scattering representation can be a 2D scattering image that can result from calculating ellipses of possible scattering positions (e.g., scan of a slice of the object 10_{slice}) and can be a 3D scattering image that can result from calculating possible ellipsoids (e.g., scan of a volume of the object 10ᵥₒₗᵤₘₑ). In some embodiments, a 2D scattering image can result from combining or considering a plurality of 2D scattering images of a same 2D area of the scanned object resulting from various sub-acquisitions completed from various source positions and detector positions (e.g., various orientation revolution positions 51 in the plane of the scanned area). Similarly, a 3D scattering image can result from merging a plurality of 2D scattering images of various positions of the scanned object (e.g., stitching 2D images, extrapolating between 2D images, stacking 2D images, etc.) or can result from merging a plurality of 3D scattering images of various positions of the scanned object (e.g., stitching 3D images, extrapolating between 3D images, etc.) or any combination thereof.

The reconstruction method can comprise an image reconstruction step, which can represent the determined arcs of possible scattering locations in a scattering representation, here illustrated as observed from a field of view 300 within which the scattering representation can be imaged, as illustrated in FIG. 3B that represents the superimposed arcs 101a/101b of the example of FIG. 3A. In some embodiments, the working region 44 may not be defined and larger arcs or the entire ellipse of possible scattering positions 100a/100b may be used for the reconstruction of the scattering representation.

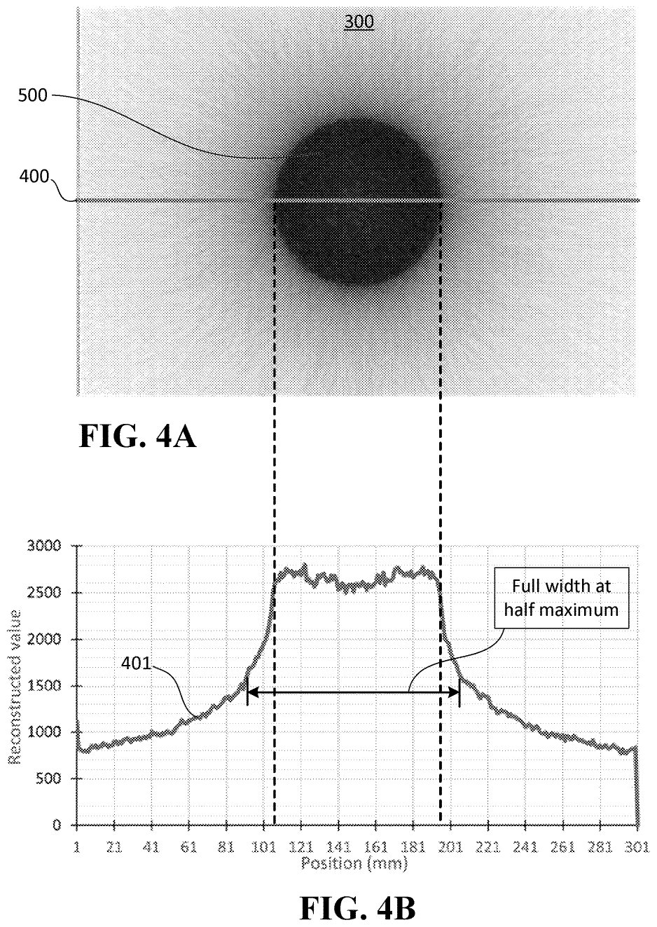

In most cases, a plurality of scattered photons may be detected for a given X-ray acquisition at a given orientation around the object of interest. FIG. 3C illustrates a reconstructed scattering representation 300′ of a single computed tomography scan, where multiple scattered photons were detected and considered to each having been scattered once. When multiple projections are acquired from various orientations around the object, and their determined ellipses/arcs of possible scattering positions are combined, added or superimposed, a resulting reconstruction of the scattering representation (e.g., image of scattering) can look like FIG. 4A.

It will be appreciated that the embodiment of the 2D case (e.g., fan-shaped X-ray beam and 1D detector array) described above that calculates and uses ellipses can be considered to be a specific case of calculating ellipsoids of a computed tomography scanner having a planar (two-dimensional) working region comprising the source and the 1D detector array, where the ellipses can be a 2D cross-section of the corresponding ellipsoids.

In embodiments where the X-ray beam is a fan-shaped beam and the detector is a 2D detector array, the working region generally dictated by the beam shape may still be considered as a planar working region (e.g., plane of the fan-shaped beam). Therefore, the arcs of possible scattering locations can be calculated by considering the cross-section of the three-dimensional ellipsoids intersecting with the planar working region (e.g., the plane of the fan-shaped beam), that may be calculated for each of the scattered photons detected by the 2D detector array. In other words, in such embodiments, the arcs of possible scattering locations used for the reconstruction of the scattering image can be defined by the portion of the ellipsoids comprised in the plane of the 2D beam.

In embodiments where the X-ray beam is a 3D cone-shaped beam, the working region generally dictated by the beam shape may be considered as a corresponding 3D cone-shaped working region. In such embodiments, whether the detector is a 2D or 3D detector array, the areas of possible scattering locations may be considered to be portions of the ellipsoids of possible scattering positions, that may be calculated for each of the detected scattered photons, comprised within the 3D working region. It will be appreciated that a 1D detector array may not limit the possible locations of scattering to an ellipse and can be used to provide ellipsoids of possible scattering positions if the working region (e.g., the shape of the X-ray beam) allows it.

While the proposed method can be completed by determining the portions of the ellipsoid's surface of possible scattering location within the working region 101 for each individual detected scattered photon 36 based on the position and the time-of-flight, it will be appreciated by someone skilled in the art that this same concept based on the position and the time-of-flight of detection can be transposed more generally, and potentially more efficiently, to the time-of-flight distribution of detected photons for each detector cells 31 of the detector 30 to simultaneously process all scattered photons detected at a same time by a same detector cell or a selection of detector cell (e.g., all detector cells having a same distance from the X-ray source). More generally, it can be understood that any suitable similar or alternative simultaneous processing of a plurality of detection data (e.g., a plurality of time-of-flights) may be used to speed up the imaging method.

FIG. 3D, which illustrates various portions of various ellipsoids of possible scattering locations 101, 101′ and 101″ (which can be limited within the working region 44 of the device) as a function of the times of detection 333, shows an embodiment of the proposed method that can utilize the time-of-flight distribution 313 of a detector cell to consider all the scattered photons detected at a same time to have the same ellipsoid of possible scattering locations. In such embodiments, for each detector cell 31 that can be associated to a single detection location on the detector 30, the portion of the ellipsoid of possible scattering locations 101 can be predetermined for each increment of times of detection 333 of the scatter portion 303 (between a time-delay threshold 16 and a cut-off time 18) of the detection curve 313 since they can correspond to various time-of-flights. Then, when constructing the scattering representation, each portion of the ellipsoid of possible scattering locations 101 can be simply multiplied by the associated number of scattered photons detected at the corresponding time (time-of-flight detection count).

FIG. 4A shows a reconstruction of the scattering representation 300 resulting from a simulation of the proposed method and apparatus. Here, the Monte Carlo simulator GATE, modified to disable absorption and only enable Compton scattering physics to generate scattered photons, was used to simulate a system that considered an embodiment having a 100 keV monoenergetic X-ray point source and a cylindrical object (e.g., phantom) having a radius of 90 mm. The reconstructed object 500 can be seen as emerging from the superimposition of the ellipses of the scattering representation 300. The distribution of the number of interactions 401 (superimposed ellipse arcs) as a function of the position along the line profile 400 displayed in FIG. 4B can show that while the full width at half maximum is of about 105 mm (about 17% larger than the simulated object), the width between the significant drops of the number of interactions (i.e., within the dashed lines) is of about 90 mm which corresponds to the simulated cylindrical object.

It will be appreciated by someone skilled in the art that, when the scanning of an object involves the rotation of the scanning components around the object, which can result in regions of the scanned object being subjected to a non-uniform quantity of photons, the count or weight of a pixel or voxel (e.g., of the reconstructed image) can be adjusted for the geometry of the scanner. This can be done by compensating for some regions being scanned more frequently (e.g., as a function of the radial position of a pixel/voxel from the rotation axis) or more densely (e.g., as a function of the shape and/or the density distribution of photons—the closer the pixel/voxel from the source the higher the density of photons), as it is usually done in the art for conventional CT scans, for example.

It will be appreciated that a similar result can be obtained with a single scan and without requiring a rotation of the measurement apparatus if a conic beam and a 2D detector are used, which would comprise a plurality of overlapping ellipsoids.

Various algorithms (e.g., programs and/or artificial intelligence) can be used to complete some embodiments of the proposed reconstruction. So far, the proposed image reconstruction algorithm is similar to a basic back projection, the simplest algorithm known in the literature. The filtered back projection (FBP) already demonstrates some improvement over the back projection by removing low frequency components in the image and increasing the high frequencies. This leads to sharper images with a better contrast to noise ratio. Similarly, other reconstruction algorithms can use a model of the scanner (named matrix system) to reconstruct iteratively the image (e.g., using at least one iterative method that can be an iterative algorithm, an iterative algebraic method, or any suitable iterative process) or can use artificial intelligence. For standard CT, such algorithms have demonstrated their superiority over non-iterative algorithms such as FBP and could further improve the image quality. Although better image quality can be obtained, the back-projection algorithm presented above already demonstrates the ability to reconstruct scatter images based on the time-of-flight of singly scattered photons (photons being scattered once between the source and the detector).

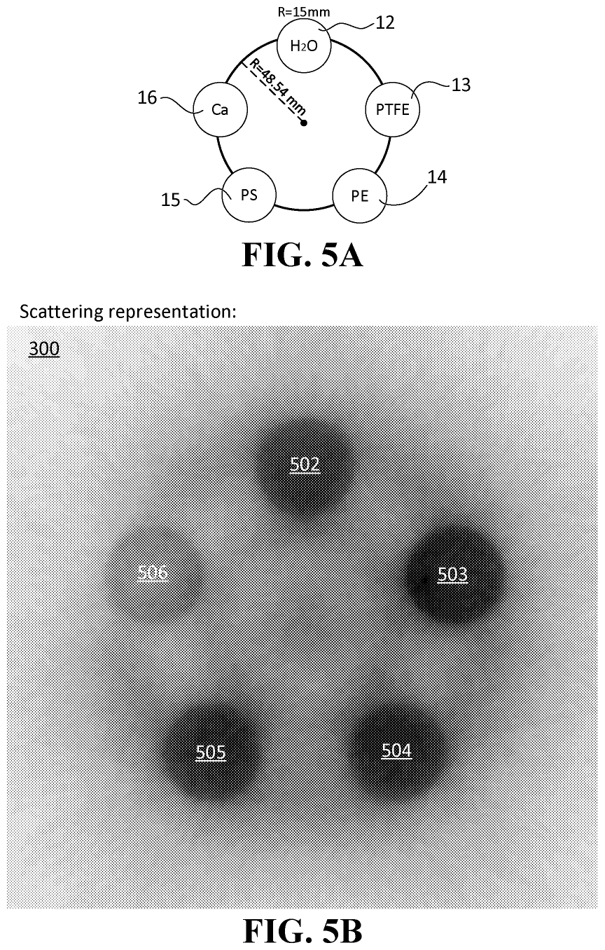

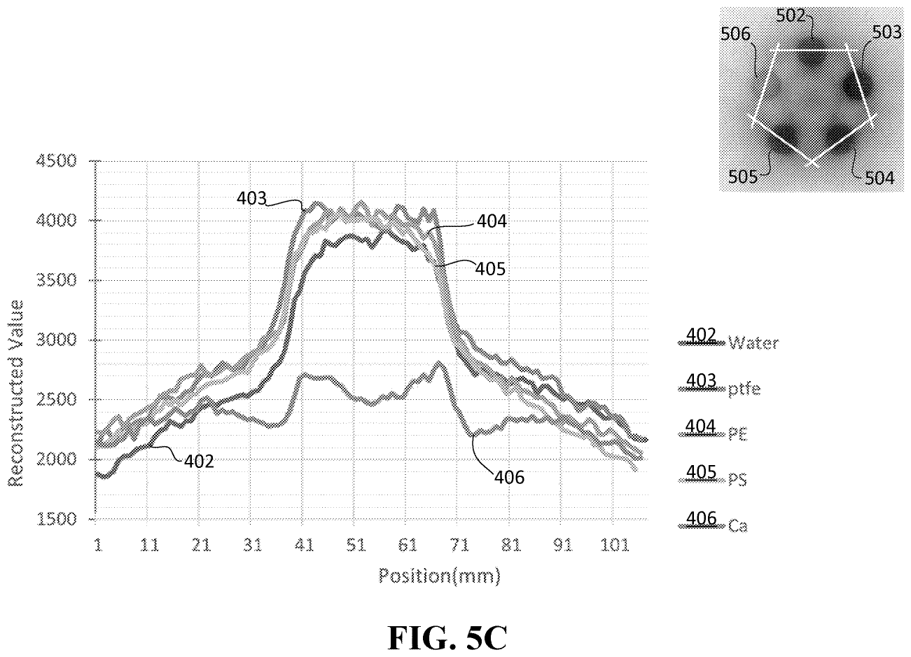

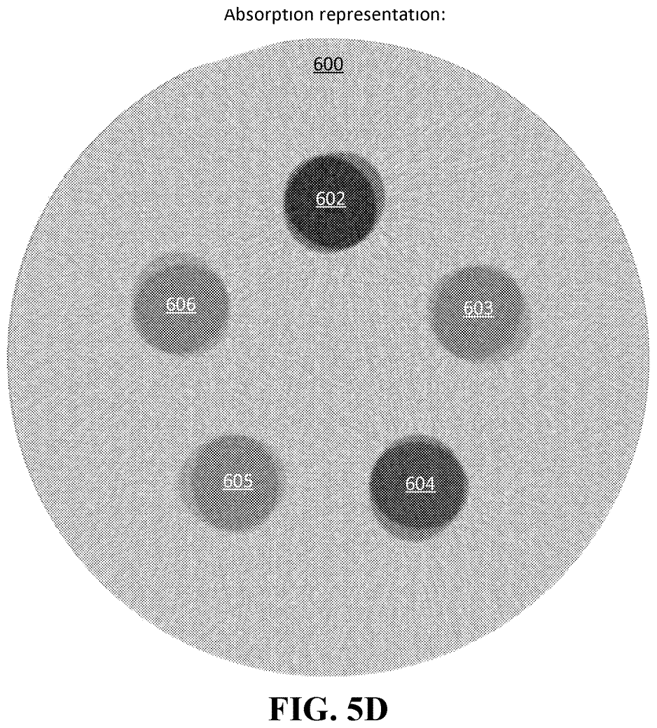

FIG. 5A is a drawing of an arrangement of various simulated inserts 12, 13, 14, 15 and 16 in a phantom, each insert having a radius of 15 mm and made of different materials: water (H₂O), polytetrafluoroethylene (PTFE), polyethylene (PE), polystyrene (PS) and calcium (Ca), respectively. Using simulations, FIG. 5B depicting a scattering representation 300 is obtained. For each of the inserts enumerated above, a corresponding reconstructed insert can be identified; 502, 503, 504, 505 and 506, respectively. FIG. 5C shows line profiles 402, 403, 404, 405 and 406 through each of the reconstructed inserts appearing in FIG. 5B corresponding to the number of interactions (superimposed ellipses) as a function of position along the corresponding line segments illustrated as white lines crossing the various reconstructed inserts in the top-right corner of FIG. 5C.

The proposed method can include identification of a material that is part of the scanned object. The material may have a corresponding electron density, which can be proportional to the number of scattering events that can be observed of a given X-ray beam passing therethrough. The results of the reconstructed scattering representation can therefore be used to approximate the electron density of a scanned object or of parts thereof to determine the corresponding material. For a given X-ray source intensity, each material in FIG. 5A produced a different number of scattering events. It may be possible to calibrate the apparatus using various objects of various materials and define reference values for a given apparatus and various X-ray source intensities, which can give a specific reference value of the number of scattering events for the specific parameters and possible setups of the apparatus (e.g., shape of the beam, type of source, type of detector, beam intensity, source/detector distance, etc.). For a given apparatus and setup, the corresponding reference value of numbers of scatterings can be used and compared to the measured values of a scan result to calculate the relative values (e.g., normalized values).

In the simulation results presented in FIGS. 5B and 5C, water is considered as the reference value for the number of scattering events and the number of interactions can be normalized to the number of interactions for the phantom made of water to get the ratios compiled in the table below (column scattering interactions). The material of each of the simulated inserts (FIGS. 5A and 5B) have distinct electron densities; 3.34×10²³ electron/cm³ for H₂O, 6.3×10²³ electron/cm³ for PTFE, 3.3×10²³ electron/cm³ for PE, 3.43×10²³ electron/cm³ for PS, and 4.81×10²³ electron/cm³ for Ca. Electron densities per unit mass (Nₑ) may be calculated using the effective atomic number (Z_{eff}), the atomic mass (Aᵢ), the Avogadro constant (N_{A}), the number of atoms (n), that can be found in the periodic table of elements and the equation Nₑ=N_{A} (n*Z_{eff})/Σᵢnᵢ*Aᵢ and the electron density per unit volume (N′) can be calculated with N′=Nₑ×ρ, where ρ is the volumetric mass density of the material. The electron densities can be normalized using the electron density of water to get the ratios compiled in the table below, which can be compared to their corresponding scattering interaction ratios.

The simulations can also demonstrate that the number/count of obtained/detected/determined scattering interactions can then be compared to the electron densities, since they are proportional.

| Material | Electron density ratio | Scattering interactions ratio | Gap |
|---|---|---|---|
| H₂O | 1.00 | 1.00 | — |
| PTFE | 1.89 | 1.10 | 42% |
| PE | 0.99 | 1.08 | 9.1% |
| PS | 1.03 | 1.06 | 2.9% |
| Ca | 1.44 | 0.72 | 50% |

It will be appreciated by the person skilled in the art, that such scattering representation and/or scattering interaction ratio

The proposed method can successfully generate ellipses or ellipsoids corresponding to the possible scattering event locations of photons scattered once. These ellipses contain information about these locations since each ellipse passes through these locations. Superimposing the ellipses can also contain enough information for locating matter with different properties, enabling to recreate the shape of simple inserts placed in phantoms. The deviation of the dimensions between those of the real insert and those of the reconstructed insert (e.g., diameter of a cylinder) may partly be due to statistical noise similarly to what is seen in PET and can potentially be corrected with an appropriate algorithm.

The simulations presented herein suppose a perfect X-ray pulse and a perfect timing of the detector which may not be achieved with empirical setups. In some embodiments, the point of emission and the depth of interaction within a detector may be taken into account, where the detector timing, depth of interaction and emission point of X-ray can generate some timing jitter that may be considered and may result in thickening the ellipses/ellipsoids.

It will be appreciated that, in practice, the photons absorbed (before and/or after the scattering events) by any object during an acquisition cannot be detected as a scattered photon. Therefore, the absorption coefficients of the various objects being scanned (i.e., X-rayed) may affect the accuracy and/or precision of the scatter measurements.

Furthermore, in some embodiments, the presence of fluids (e.g., water) between regions with different material properties within the scanned object or the proximity of different materials may be detected since it can influence the precision of the results and, in some case, may lead to a reduction of the quality of the scattering representation.

Further signal filtering or signal processing can be considered and added to the proposed method to improve the images obtained. A relationship can also be established between the angle of the scattering and the ellipse eccentricity so that a smaller scattering angle can result in a higher ellipse eccentricity (e.g., for a fixed distance between the two foci, this means a shorter minor axis of the ellipse).

In some embodiments, the detector array can be placed at a different angle from the direction of the X-ray beam, which may result in a larger ellipse eccentricity or a change of the working region. This may push one side of the ellipse outside of the object and reduce the noise level. The relative number of scattering events generated by different materials can be different from the relative numbers of photons absorbed by the photoelectric effect in the same materials. A correct reconstruction of the scattered photons in computed tomography may lead to new ways of identifying the composition of the object being imaged.

In some embodiments, photons may be discriminated in various images or within a color-coded representation, according to the difference between the straight-line distance of the source to the detector and the distance (length) traveled by the photons that can be calculated with the time-of-flight and the speed of light. A larger difference in these distances may mean that the photon had a scattering event occurring at a greater angle. In some embodiments (e.g., in a real system), a larger difference can also imply a higher probability that the photon scattered multiple times. In some embodiments, different images or representations might contain complementary information on the materials.

The proposed method may be used with various computed tomography mechanical setups or apparatus. In an embodiment, an apparatus comprising an X-ray source 20 for generating a pulse of X-photons, a time-sensitive photodetector (e.g., detector 30) and a processor may be required to functionally use the proposed method.

FIG. 5D presents a simulated absorption representation 600 of a scan of cylindrical phantoms illustrated in FIG. 5A. The simulated absorption representation 600 comprise five various shapes that can be distinguished from the background, namely an absorption representation of a mass of water (H₂O) 602, an absorption representation of a mass of polytetrafluoroethylene (PTFE) 603, an absorption representation of a mass of polyethylene (PE) 604, an absorption representation of a mass of polystyrene (PS) 605, and an absorption representation of a mass of calcium (Ca) 606. The simulated absorption representation 600 may be used to better appreciate the various differences that distinguishes a scattering representation 300 and an absorption representation 600. The person skilled in the art can appreciate that the ballistic (absorption) information that may be provided by and/or extracted from the absorption representation 600 can complementary to the scattering information that may be provided by and/or extracted from the scattering representation 300, which may be used in combination to help identify more accurately and/or with more precision the nature of a scanned object.

For example, in this specific exemplary embodiment, while the calcium 16 of FIG. 5A shows up as the lowest scattering medium (that corresponds to the lighter circle 506 in FIG. 5B) out of all the mediums of this simulation, the calcium 16 may seem to have an absorption coefficient (illustrated in FIG. 5D as the absorption representation of a mass of calcium 606) similar to the absorption coefficient of the polytetrafluoroethylene 13 and of the polystyrene 15 (illustrated in FIG. 5D as the absorption representation of a mass of polytetrafluoroethylene 603 and of polystyrene 605). Therefore, an object made of calcium may be identified by having both relatively low absorption and scattering coefficients. This may be particularly useful, accurate and precise when considering quantitative values.

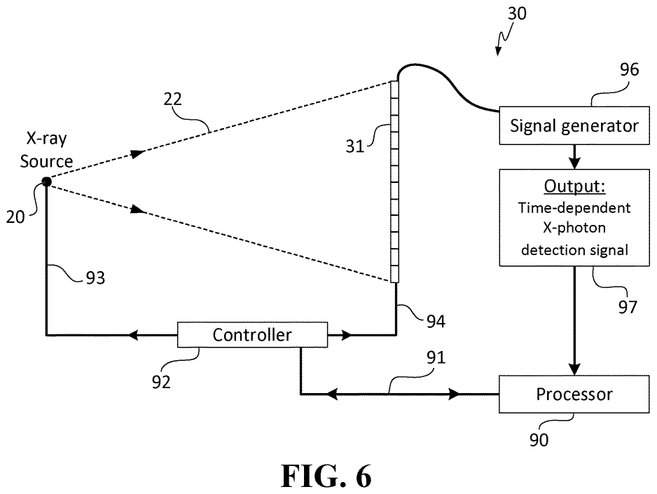

FIG. 6 presents a schematic drawing of an embodiment of a proposed apparatus comprising a processor able to perform the steps of the proposed method. It will be appreciated that this embodiment is in such detail as to clearly communicate the disclosure without limiting the anticipated variations of the possible embodiments and may encompass all modifications, equivalents, combinations and alternatives falling within the spirit and scope of the present disclosure. It will be appreciated by those skilled in the art that well-known methods, procedures, physical processes and components may not be described in detail in the following so as not to obscure the specific details of the disclosed invention.

The proposed apparatus can comprise a processor 90 that can send instructions to a controller 92 that can send control signals to a source 20 for generating a pulse of X photons 22 (X-ray beam) and/or can send controls signals to a time-sensitive photodetector 30 for measuring/detecting the X photons emitted by the source. In some of the preferred embodiments, the processor 90 can be an integrated circuitry that may be a central processing unit (CPU). In an embodiment, the controller 92 can send a control signal to the source 20 to request it to generate a pulse of X photons and can send simultaneous or delayed control signals to the detector 30 to activate/deactivate the photodetector. In an embodiment, the processor 90 can act as the controller 92. In some embodiments, the proposed apparatus can comprise a signal generator 96 that may generate a signal including data/information about the detected photons such as their position on the detector array (detection location) and their time-of-flight. In an embodiment, the signal can be a time-dependent X photon detection data/signal 97 (e.g., comprising a plurality of time-dependent values and a corresponding detection location on the detector array) that may be sent to the same processor 90 or to an alternative processor 90′ to be further analyzed and processed. In some embodiment, the controller 92 can be embedded in the X-ray source 20.

It will be appreciated that the processor 90/90′ can comprise or can be connected to and be used in combination with a proper input interface and suitable memory as known in the art. The memory of the device (e.g., X-ray imaging apparatus) can be used to save any relevant data (e.g., any data acquired or generated by the processor 90/90′ or any signal generator 97) and/or to store at least one program code (program instructions and commands) to properly operate the device and/or any other type of required information. In some embodiments, the memory can be any suitable type of transitory and/or non-transitory memory known in the art, which may be at least one of: random-access memory (RAM), read-only memory (ROM), solid-state drive (SSD), hard disk drive (HDD), a combination thereof, etc. In some embodiments, the memory can comprise a plurality of memory layers.

A signal generator can comprise circuitry or a program for converting the various signals (e.g., trigger signal of the X-ray source, photon detection signals of the detector's pixels, etc.) to a digital value (e.g., time-of-flight of detected photons, timestamps, counts, detection locations, etc.) which may be stored into a database and/or converted into detected photons datasets. To do so, the signal generator 96 can comprise, for example, a time-to-digital converter (TDC) which can be, but is not limited to, the embodiments of a TDC described in the PCT application WO2021243451A1, published 9 Dec. 2024. In some embodiments, the generated digital values can be stored as measured timestamp data which can comprise a timestamp matrix collecting, for each location of the various detector cells 31, each of the measured time-of-flights (timestamps of the photons detected by each detector cells 31).

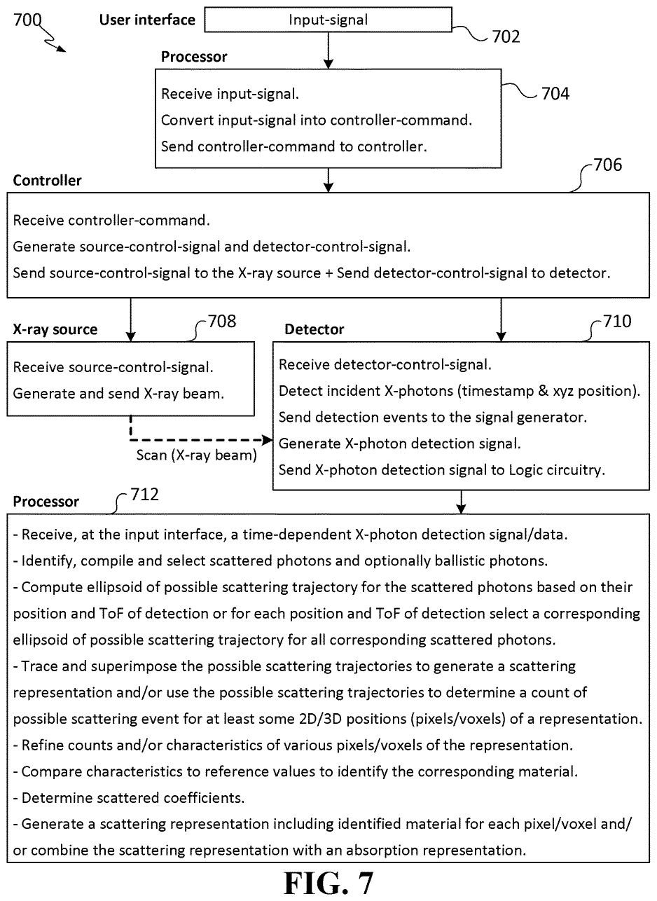

FIG. 7 shows a block diagram 700 of various steps that may be performed by various components of an embodiment of a computed tomography scanner that can perform the proposed method. In some embodiments the apparatus can comprise a user interface 702 that can be used by an operator to engage the X-ray imaging process by generating an electronic or mechanical input signal. In some embodiments, the apparatus can comprise a processor 90 for performing at least some of the processor's steps 704 that can be used to send a controller-command to the controller 92 for performing at least some of the controller's steps 706 upon receiving an input-signal. In some embodiments, the apparatus can comprise a controller that can generate a source-control signal to send to and to control an X-ray source 20 for performing at least some of the source's steps 708 and can generate a detector-control signal to send to and to control a detector 30 for performing at least some of the detector's steps 710 upon receiving a controller-command signal or alternatively upon receiving an input-signal. In some embodiments, the apparatus can comprise a processor 90′ for performing at least some of the various steps 712 that can be completed by a processor and/or an imaging apparatus that can include, but not limited to, the ones enumerated in the last block 712 of the bock diagram of FIG. 7. In some embodiments, a processor can comprise both processors 90 and 90′ or can perform their associated steps 704 and 712 as described above and in FIG. 7.

In some embodiments, the processor may be configured or programmed to operate the necessary steps to perform the method proposed herein. Some of these steps may be some of the steps that can be performed by the proposed apparatus in order to execute the method proposed herein, which may include: receiving an input signal from an operator/user; converting this input signal into a controller command; sending this controller command to a controller 90; receiving an X photon detection signal from the signal generator 97 that may be included in the detector 30; identify, compile and selecting the detected scattered photons and optionally ballistic photons from that detection signal; computing and calculating coordinates of possible elliptic/ellipsoidal scattering trajectories for each selected scattered photons, or for each detection location and corresponding time-of-flight select a corresponding ellipsoid of possible scattering trajectory for all corresponding scattered photons; trace and superimpose the possible scattering trajectories to generate a scattering representation and/or use the possible scattering trajectories to determine a count of possible scattering events for at least some 2D/3D positions (pixels/voxels) of a representation; refining, identifying, and extracting some of the characteristics of the various pixels/voxels from the resulting scattering representation using one or more of the possible correction methods; comparing these identified characteristics to reference values to identify the corresponding material; determining scattered coefficients of the various structures of the scanned object and of the various pixels/voxels; and generating a more complete scattering representation that may include the identified material of some of the pixels/voxels and/or combine a scattering representation with an absorption representation of the corresponding pixels/voxels. It will be appreciated that some of these steps may not be required to complete some embodiment of the proposed method.

### Data Refinement and Correction Methods

It will be appreciated that an object can be comprised of a wide variety of structures and arrangements of structures that can each have their own complexity and characteristics. In fact, living organisms, for example, can be very complex structures comprising a wide variety of complex materials/tissues. Therefore, a non-negligible portion of the incident photons of an X-ray source are likely to interact with such objects in various ways before and/or after they are potentially scattered.

It may not be sufficient, therefore, to assume that all regions of such objects are uniformly subjected to a uniform number/density of incident photons. For example, a region (say first region) of the object could have a high absorption coefficient, which could block or prevent a significant portion of the incident photons from reaching another region beyond the first region (on the path of the photons) of the object. In other words, some structures or regions of an object could induce a “shadow”, effectively reducing the density of photons traversing the shadowed regions or structures which could be less likely to interact with photons, thus reducing the absolute number of scattering events in the shadows, consequently reducing the absolute number of detected scattering events in the shadowed regions, and reducing the accuracy and precision of the characterization of the shadowed region.

When absorption (e.g., photoelectric absorption) occurs in a material, the reconstructed relative values of the scattering coefficient may have an accuracy-discrepancy, meaning that they may no longer be as close as they should to the relative values of the electron density. Even if a proportion of photons is scattered by Compton scattering, a part of these photons may be absorbed before reaching the detector, which may lead to a lower number of ellipses/ellipsoids, reducing the number of recovered scattering interactions. This may explain larger discrepancies with the expected values. In other words, the accuracy of the reconstructed values can be influenced by a possible subsequent absorption and/or Compton scattering of the photon. This can be analogous to state-of-the-art computed tomography, since scattering can also influence the number of transmitted photons in absorption images. In addition to this, unlike the monochromatic source considered in the simulations, a polychromatic source can emit photons at a lower energy, which may be more frequently absorbed. Thus, an appropriate correction could preferably be applied to the data to recover as accurately as possible the true relative values of the scatter coefficient in the material.

It will be appreciated by someone skilled in the art that, an accuracy-discrepancy between the “raw” (uncorrected) results and the expected (real) values may also be a result of a decrease in the number of incident photons reaching a given region of the scanned object (e.g., a region having this accuracy-discrepancy).

It will be understood by someone skilled in the art that the shadows are most likely to reduce the density of incident photons in the shadowed-subsequent-region, which is most likely to reduce the total number of scattering events, which would result in a reduced number of scattered photons being detected. In fact, while the probability for photons to be scattered and the relative portion of photons being scattered may remain unchanged, the total number of scattering events would decrease if the total number of photons (photon density) decreases. Such a reduction in the total number of detected scattered photons originating from a shadowed region is likely to be reflected on the uncorrected values as being a region wrongfully characterized as being less scattering. Therefore, some embodiments can use a correction method to correct for this phenomenon.

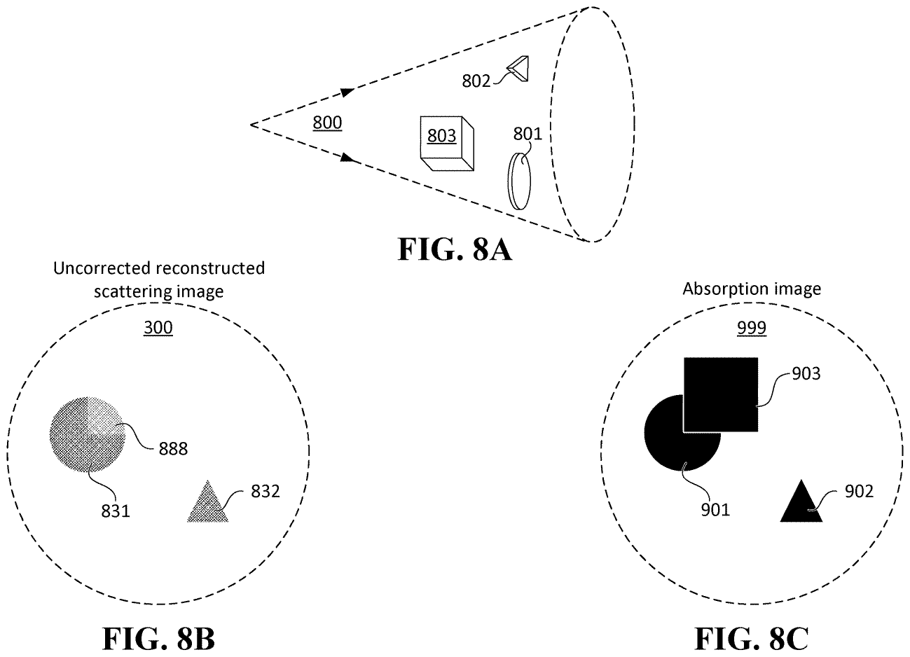

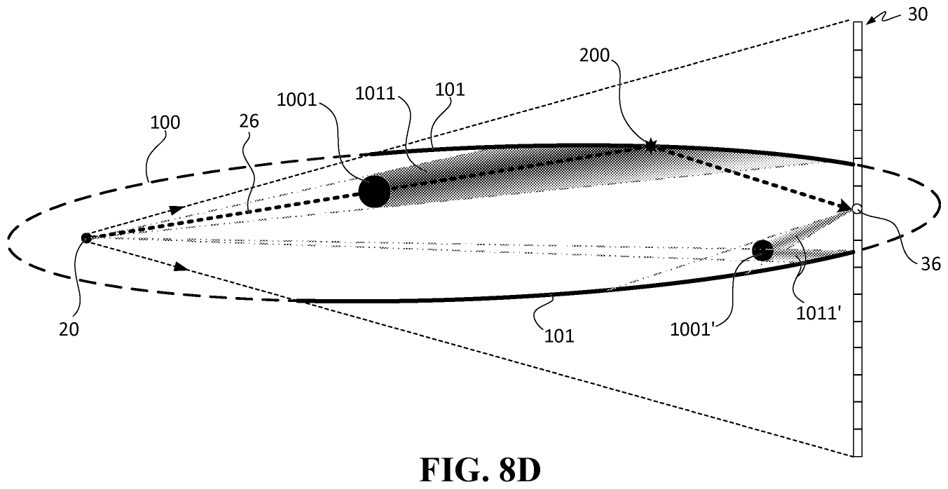

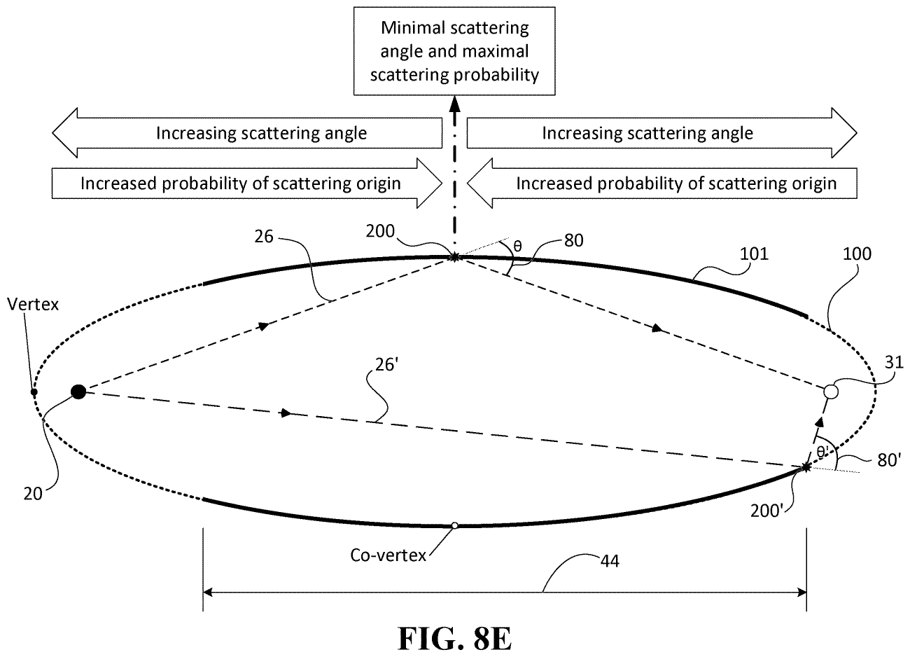

FIG. 8A illustrates a schematic drawing of a perspective view of a cone-shaped beam 800 used to scan an arrangement of three objects: a first object 801 having a circular cross-section, a second object 802 having a triangular cross-section, and a third object 803 having a square cross-section. In this example, this arrangement of objects is scanned so that the measurement of detected photons can be used to generate both an absorption representation and a scattering representation. It will be noted that, in order to simplify the drawings and for the purpose of illustrating this “shadow” effect, the third object 803 absorbs a portion of the photons traversing its structure without inducing any scattering of photons.

FIG. 8B illustrates a scattering representation 300 corresponding to an uncorrected reconstructed scattering image of the arrangement of FIG. 8A. This reconstruction can be generated using the method previously detailed and from the data of scattered photons selected from this measurement of detected photons. This reconstruction comprises two scattering objects; a first scattering object 831 corresponding to the first object 801 and a second scattering object 832 corresponding to the second object 802. In this exemplary uncorrected reconstructed scattering image, a quarter 888 of the first scattering object 831 appears to have generated less scattering events, which could be interpreted as a region of the first object having a lower scattering coefficient if the measurements remained uncorrected.

FIG. 8C illustrates an absorption image 999 generated from the detected ballistic photons, where the first object 801 appears as a first absorbing object 901, the second object 802 appears as a first absorbing object 902, and the third object 803 appears as an absorbing object 903. It will be appreciated by someone skilled in the art that the absorbing object 903 of FIG. 8C may most likely be responsible for the quarter region of less scattering 888 of the first scattering object 831, since this absorbing object 903 most likely reduced the number of incident photons that reached the first scattering object 831 which would inevitably reduce the number of scattering interactions in this region. This simplified example illustrates the concept of a shadow of photons produced by an absorbing object, here the shadow from the third object 803, on a scattering measurement that may lead to an un-exact scattering representation, here the quarter region 888 with less scattering events. Understanding this concept of a shadow of photons may be used to correct and adjust the scattering measurements to obtain a corrected measurement, to generate a corrected scattering reconstruction and/or to calculate more accurately the various relative values of scattering coefficients of the scanned objects.

Based on this concept, in some embodiments, the data and information used to generate, or extracted from, the scattering representation can be used in combination with the data and information used to generate, or extracted from, a corresponding absorption representation. Any data, measurement or information of at least one of: the detected scattered photons, the scattering representation, the detected ballistic photons, or the absorption representation can be used to adjust and/or correct the values, data, measurements or information of the scattering representation and/or coefficients.

This combination of such information and data can be used to apply a correction method to “correct” the raw (uncorrected) scattering measurements and/or results which can be used to generate a corrected (more accurate) scattering representation and/or determine corrected scattering coefficients. For example, the shadowed portions of the scanned region (i.e., having a lower scattering coefficient according to the raw uncorrected values) can be corrected considering the presence of the absorbing object identified with a complementary absorption measurement (e.g., from the ballistic photons of a same scan). In the case of the example of FIGS. 8A to 8C, this could be done by correcting the scattering measurement covered by the absorbing object 900, corresponding to the region 555, by increasing (e.g., weighting of the values or multiplying by a correction factor) the corresponding counts of scattering events and/or the corresponding scattering coefficients as a function of the absorption measurements or coefficients of the absorbing objects in the path of the scattered photons, either before the scattering interaction (between the source and the possible scattering location) or after the scattering interaction (between the possible scattering location and the corresponding detector cell).

FIG. 8D shows a 2D drawing of a different arrangement being scanned and comprising two absorbing objects 1001 and 1001′. In this example, a scattered photon is detected at a given detection location and time on the detector 30. A corresponding arc of possible scattering positions 101 (within the working region of the ellipse of scattering 100 that can be determined with the position and time-of-flight of this detected scattered photon 36) are illustrated. Note that the positions on the ellipse of scattering 100 that are outside of these arcs 101 are not considered as possible single scattering locations since they are outside of the working region (i.e., outside of the triangular-shaped beam of the source 20 or behind the detector 30). For each of the absorbing objects 1001 and 1001′, respective shadows of photons 1011 and 1011′ can be determined within the ellipse of scattering 100. These shadows can be determined by considering the arcs 101 of possible scattering positions and the characteristics of the two absorbing objects 1011 and 1011′ (e.g., their respective position, dimensions and/or absorption coefficient distribution), which can be determined from the measurement of ballistic photons. It will be appreciated that the longer the path 26 of a photon crosses the absorbing object 1001 the more likely the photon is to be absorbed, which translates in a stronger shadow of photons 1011 (i.e., lower density of photons) along the subsequent travel path 26 of this photon. This change in intensity of the shadow 1011 is illustrated in FIG. 8D as an intensity gradient within the shadow, where the shadow 1011 is darker (i.e., more intense shadow-lower photon density) when the travel path 26 of the photon traverses the full diameter of the object, see for example, the shadow 1011 of the larger object 1001 along the first part of the travel path 26 of the possible scattering events 200.

It will be appreciated that each object and structure that has a non-zero absorption coefficient can have/cast a first shadow for the first part of the scattered photon's travel path traversing the object. Therefore, the weight of the possible scattering position may simply be adjusted (e.g., increased) according to the portion (i.e., length) of the first part of the travel path of the scattered photon traversing each object and the absorption coefficients of these objects. Some of the objects and structures that have a non-zero absorption coefficient can also have/cast a second shadow for the second part of the scattered photon's travel path traversing the object. It will be further appreciated that the second shadow can vary for each different ellipsoids of possible scattering locations (i.e., for each combination of detection position/location and time-of-flight). The weight of the possible scattering position may be adjusted (e.g., increased) according to the portion (i.e., length) of the second part of the travel path of the scattered photon traversing each object and the absorption coefficients of these objects.

In other words, in some embodiments, for each possible scattering locations, the weight or count of the pixels or voxels corresponding to these locations can be adjusted considering the objects or regions on the corresponding full travel path (sum of lengths L1 and L2 as defined in FIG. 2A) of their scattered photon and using corresponding absorption measurements (e.g., position/coordinates, dimensions and/or absorption coefficient distribution of the identified objects).

In a preferred embodiment, the weight correction is proportional to the absorption coefficient of the traversed objects or regions, meaning that the longer the intersection with an object and the higher its absorption coefficient, the more the corresponding possible scattering location should be positively weighted.

It can be appreciated that the use of the absorption measurement can significantly be useful when correcting and adjusting the scattering characterization. However, such correcting and adjusting may not be limited to the previously mentioned concepts. In fact, various concepts can be used to better represent and characterize the scattering objects. For example, the position of the scattering event relative to the ellipsoid can be used to correct, adjust or weight the values or counts based on the fact that it is more probable that a scattering event occurs at a lower scattering angle.

The probability that a scattering event occurs can be determined or approximated based directly on or using an equation derived from the Klein-Nishina formula.

FIG. 8E illustrates various scattering angles 80 and 80′ associated with various possible travel paths 26 and 26′ of a scattered photon detected at the detector cell 31. In this schematic drawing, the smallest possible scattering angle 80 corresponding to a scattering event 200 located at the co-vertex of the ellipsoid, while the largest possible scattering angle 80′ corresponds to a scattering event 200′ located at the extremity of the arc of possible scattering positions 101 nearest to a vertex of the ellipsoid.

The uncorrected data (e.g., the weight and count of the pixels/voxels) can be adjusted and corrected to increase the accuracy of the associated results (e.g., corrected scattering representation and/or scattering coefficients) with this correction method that can be based on a method as previously described.

In some embodiments, a background measurement (e.g., the quantity and/or the temporal distribution of photons detected by the detector cells during a control measurement-without any object between the source and the detector) may be used to adjust the number of considered ballistic and scattered photons.

In an embodiment, the scattering representation that can comprise information about the type of material of some of the pixels/voxels may be combined with the corresponding absorption representation by color coding at least some of the pixels/voxels of the absorption representation as a function of their material type. The color coding may be a discrete color coding with each color corresponding to a given material or may be a color gradient corresponding to a scale of electron density. In the preferred embodiment, the scattering representation is a corrected scattering representation that is based on corrected data that can be corrected by one or more of the correction methods described above.

### Possible Applications

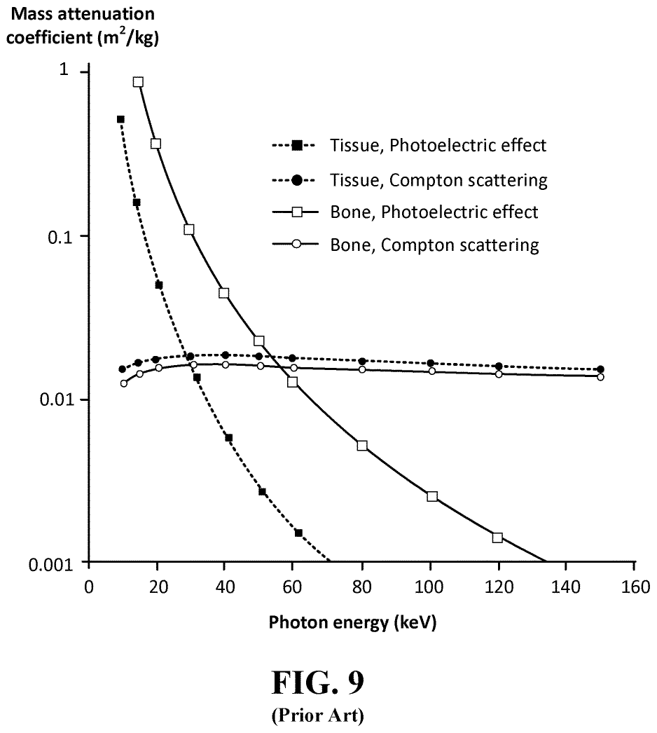

As known in the art, since human tissues have an effective atomic number Z≈7.4, the photoelectric absorption effect dominates at up to 30 keV whereas Compton scattering dominates for X-ray energies >80 keV. FIG. 9 shows schematically photoelectric absorption and Compton scattering for bones and soft tissues as a function of X photon energy in the range of 30 to 120 keV as is well known in the art. When considering the subject volume, a minimal amount of X photon energy is normally required to make an absorption image, and this can be in the range from about 60 keV to about 120 keV for humans (e.g., closer to about 60 keV for a smaller body mass and closer to about 120 keV for larger body mass) and about 30 keV to about 80 keV for small animals or breast. These ranges fall in the valley where the photoelectric effect (that provides useful information for imaging) ends and Compton scattering increases. These ranges of energy may be required to complete scans and acquisitions with the proposed method and apparatus when an absorption image is desired in addition to or in combination with the scatter image.

When considering the embodiments of the proposed apparatus that may be used to generate scattering images without measuring absorption, it may be preferable to minimize the photoelectric effect. This can be achieved by increasing the X-ray energy over about 100 keV and preferably over about 120 keV, where the probability of photoelectric absorption decreases while the probability of Compton scattering remains constant as illustrated in FIG. 9 and where tissues are of low density. It will be appreciated that the dose of photons absorbed by photoelectric effect by the object (e.g., patient) can therefore be significantly reduced when aiming at measuring a scattering image. In fact, the proposed method can be used to generate a scatter image with a negligible number of photons absorbed by the patient via the photoelectric effect, which causes little to no health risks.

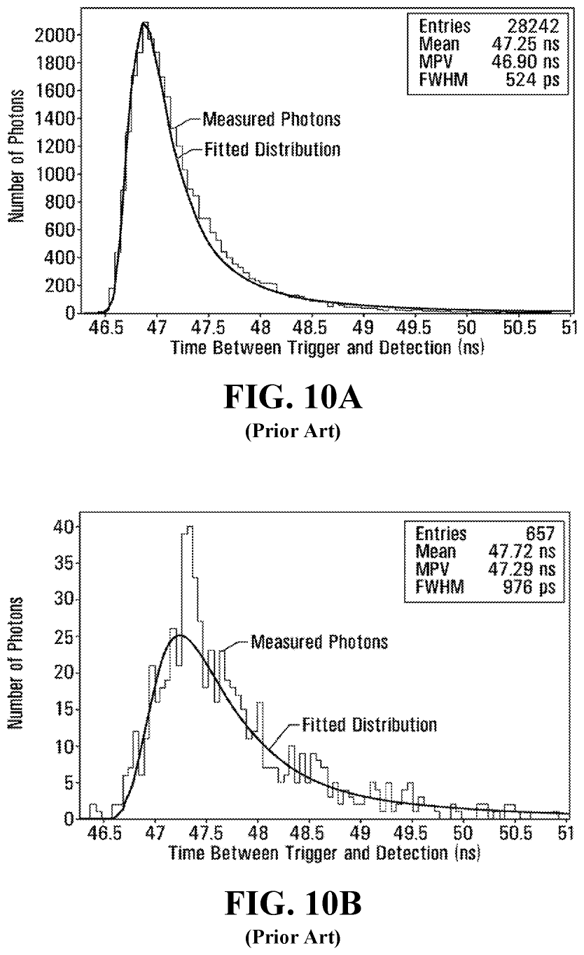

As presented in the PCT publication WO2020093140, an experiment was conducted to confirm the feasibility of observing time-of-flight differences between scattered and transmitted (ballistic) photons. In this exemplary experiment, a 3×3 mm² silicon photomultiplier (SiPM) covered by a ~500 μm thick lutetium yttrium oxyorthosilicate (LYSO) crystal was placed about 38 cm in front of a pulsed X-ray source with a mean photon energy of about 15 keV and a pulse width of about 60 picoseconds FWHM. Two measurements were made: one with nothing between the source and the detector and one with a ~40 mm thick aluminum beam blocker. FIG. 10A shows a histogram of the number of photons as a function of the time between trigger and detection (a temporal point-spread function-TPSF) without the blocker, while FIG. 10B shows the same type of histogram with the blocker. A total of 657 photons were detected after an acquisition of 36 hours. Once fitted with a Landau distribution, the most probable value (MPV) of the time-of-flight with the beam blocker is of about 390 picoseconds later than the MPV without the beam blocker, which may correspond to an increase of travel path of about 12 cm. This fits with the expected increase of travel path needed to get around the beam blocker by scattering on the X-ray enclosure. No time-of-flight correction was made for the energy of the detected photons. Higher energy photons were detected earlier owing to their steeper slope. However, the increased time-of-flight of scattered photons is observed at all energy levels at around 400 picoseconds. Dark counts and double detections were removed from both measurements.

An X-ray source can be used for emitting X-rays. A source that emits X photons of only one energy is called monochromatic (or monoenergetic), otherwise it is called polychromatic (this terminology is in analogy with visible photons for which different energies correspond to different colors-chroma). One possible means for producing X-rays can be by bending radially a beam of electrons, i.e., when the electrons accelerate perpendicular to their velocity, as, for example, in synchrotrons using bending magnets. In conventional X-ray tubes, the stream of X photons is continuous, but can be, for the purposes of the present invention, very short pulses (or bursts) of X-rays of the order of at most about tens of picoseconds as needed. Such short X-ray pulses can be generated via X-ray emission from femtosecond laser-induced plasmas on solid surfaces. Another approach can be through high-order harmonic generation in gases which resorts to intense ultra-short laser pulses, which can be carried out in gas-filled hollow fibers. It will be appreciated that these approaches can be foreseen to be amenable to reasonable sizes for integration in medical imaging devices since ultra-short pulse laser technology is nowadays highly compact.

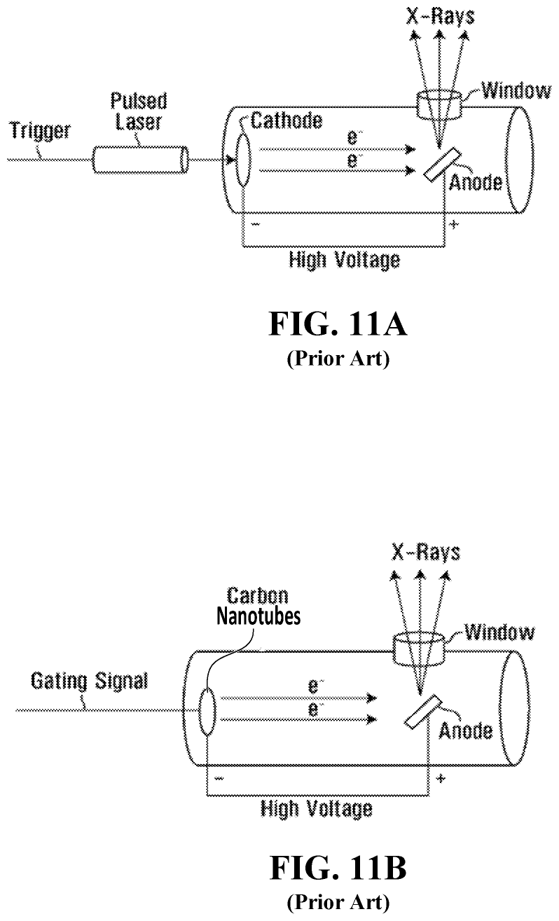

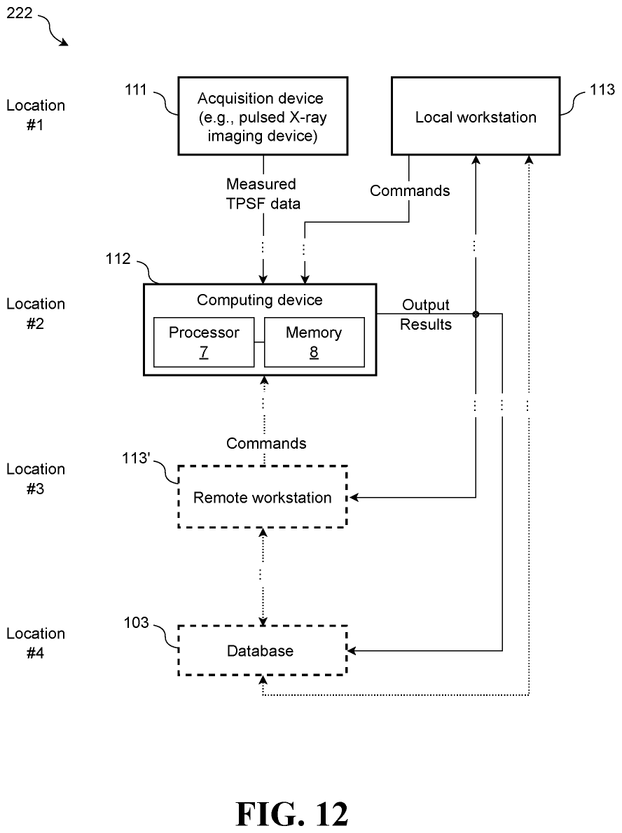

Another approach to generate ultra-short X-ray pulses is that developed for fluorescence lifetime measurements, whereby fluorescence is induced by X-ray excitation, where a pulsed laser diode emitting short pulses of light (<100 picoseconds full-width at half maximum-FWHM) are directed onto a light-sensitive photocathode that emits short bursts of electrons with each light pulse impinging onto it, as schematically illustrated in FIG. 11A. The electrons are then accelerated towards an anode as in conventional X-ray tubes described above. Yet another approach to generate short X-ray pulses may be to use an X-ray tube in which the electron beam can be very rapidly deflected as in a streak camera, with an electric pulsed field in such a way that it strikes the anode for a very short time interval in which Bremsstrahlung X-rays can be generated.

Another possible solution may be to generate X-ray pulses by replacing the photocathode with carbon nanotubes (CNT) as described in the prior art. The CNTs can be plated on top of the cathode as an electron emitter with the capability to be gated faster than the cathode alone directly with an electric signal (FIG. 11B) and operating at lower temperature.

It will be appreciated that different technology configurations can support embodiments of the invention and are not limited to the examples described herein.

The detector is among the important components to consider in the deployment of the proposed technology. There can be two main detection principles: direct conversion and indirect conversion. While the direct conversion of X photons in materials such as semi-conductors, germanium or silicon is very attractive for high energy resolution, indirect conversion can be a preferred avenue nowadays thanks to its lower operating voltage and its proven better timing resolution. The use of a thin scintillator able to stop an X photon coupled to a high-speed photodetector such as a silicon photomultiplier (SiPM) or any of its digital derivatives may be a good candidate for a complete system with timing performance under about 1 nanosecond and preferably about 200 picoseconds. The time-sensitive detector can be used to determine and identify the three-dimensional (x, y and z) detection location of each detected photon. In some embodiments, the z coordinate can correspond to a depth of interaction with the detector, which may be of a same value for all xy coordinates (e.g., when a detector having a single thin semi-conductor layer) or can have various values (e.g., as in a scintillator or when a detector having a stack of thin semi-conductor layers).

Although an indirect conversion mechanism can be preferred, it may be intimately coupled to an adequate gating mechanism. The brute force approach could be to timestamp every individual photon and to provide the information to a digital signal processor able to process, in real-time, the relevant information for image reconstruction. This processor could be integrated in 2.5D or 3D electronics along with the photodetector or be located remotely outside the scanner. The processing algorithms can be of any form from gating, filtering, up to machine learning. However, this approach will require a large data bandwidth and other approaches can be used.

In order to reduce the bandwidth, an adjustable and delayed trigger can be distributed in the scanner. This trigger can open a time window where all photons striking the detector in the time window are timestamped or counted. The information can still be sent to a local or a remote digital processor with the goal to extract the relevant information to be fed to the image reconstruction algorithm. The trigger can be self-adjusted from the center of the detector panel to the periphery or manually adjusted with programmable or fixed delay lines to take into account the source to flat panel distance variation from the center to the periphery. In the former case, each pixel has a communication link with its adjacent neighbor while in the latter case, a system calibration can be mandatory.

It would also be possible to use energy integration detectors to increase the time resolution of the detection by applying a correction factor to the measured time-of-flight. In some embodiments, however, the resolution of the energy detection may not be significant, especially when using X photons.

Optimizing time-of-flight X-ray imaging (whether 2D or 3D) revolves around one central idea: having the right time window width (i.e., timespan, duration or time interval). Ideally, the time window (time gate) width should be selected to measure almost all ballistic photons and send this information to the image reconstruction engine dedicated to absorption measurement and then keeping the maximum number of singly scattered photons to create a scattering image able to quantify scattering coefficients. To achieve this, the impact of every component in the system on the time window width may preferably be considered. Component or subsystem specifications that widen the response of the system to ballistic photons lead to uncertainty on the measurement of time-of-flights. Such specifications are, for instance, the pulse width of the X-ray source (and in some cases the sharpness of the rising edge of the X-ray pulse), the timing resolution of the detectors or time threshold distribution within the system. To keep almost all singly scattered photons, the time window can be widened when time-of-flight measurement uncertainty increases. However, doing this may reduce the proportion of ballistic photons being sent to the image reconstruction algorithm. Since removing part of the singly scattered photons reduces the contrast-to-noise ratio (CNR) while removing more ballistic photons increases the CNR as regards the scattering image, the width of the time window can be chosen more conservatively or more aggressively, according to whether the CNR of absorption or diffusion is the parameter of importance.

On one hand, increasing the signal-to-noise ratio can be particularly important in very low dose applications and in imaging systems with a naturally high contrast such as for inorganic imaging. On the other hand, improving the CNR requires a higher dose in imaging biological tissues characterized by small electronic density differences such as breast tissues in breast imaging.

Improving the total timing resolution by reducing it is one of the most important design aspects of time-of-flight X-ray imaging systems (whether 2D or 3D). The timing uncertainties caused by the different system components are added together in quadrature. Thus, reducing the pulse width and the timing resolution of the detectors can be equally important to increase the efficiency of discrimination. If both effects are reduced to under 10 picoseconds, the spatial uncertainty of the emission (the size of the focal spot of the source) and of the detection (size of the detector and error on positioning) can also be optimized since these affect the measured time-of-flight for ballistic photons used as a comparison for the discrimination. Jitter between detectors will also increase the error on the measurements and will have to be reduced to a minimum.

Embodiments can be implemented in a variety of systems. The following are potentially interesting applications of time-of-flight radiography and computed tomography:
Pediatric imaging (provided the radiation dose can be reduced to acceptable levels);
Preclinical imaging, e.g., small animal imaging;
Dental care (where the dose and the form factor are important);
Bariatric patients (where the CNR is normally a problem);
Extremities (the form factor and the dose);
Interventional radiology (the form factor, the dose and the resolution);
Gated imaging, such as in cardiac or respiratory gated imaging;
Chest imaging (CNR is important due, in part, to the thickness)

Scatter imaging may allow to better identify some materials with the same dose such as in the following applications:
Oncology (provide a measurement of iron in the tumor and determine its degree of vascularization)
Inflammation (provide a complementary image of vascularization)
Contrast agent (provide a finer distribution of contrast agents in blood vessels, inside the gastrointestinal, biliary or urinary tract, blood flow in organs)
Contrast agent (new contrast agent not based on heavy materials can now be used)
Tissue characterization (such as in soft tissues, fat, tendon, and bone)
Bariatric patients (where the X-ray photon energy may have to be increased and the probability to create a scatter may increase. CNR of scattering imaging should be improved);

Outside the medical field, scatter imaging can be used in:
The food processing industry (determining contaminants)
Safety (detecting explosive or narcotics)
Any time gated scatter imaging enabled in a light source facility, for example.

It will be appreciated that the information emerging from the absorption and scattering imaging/analysis can be extracted from a single scan of the object, which is one of the main advantages of such a combination of information. In fact, since the ballistic photons used for absorption imaging and the scattered photons are present within each scan and can be effectively differentiated from one another with their time-of-flight, both set of detected photons can be acquired with the same acquisitions of a scan.

It will be appreciated by someone skilled in the art that the same X-ray pulse can be used to acquire both the absorption and scattering images without adding an extra dose to the patient.

Spatial resolution is particularly important for both pre-clinical and interventional radiology. Higher spatial resolution calls for small pixel detectors and reduced noise in the image requires high time resolutions. Both can be obtained by using embodiments as described herein in conjunction with single photon avalanche diodes (SPAD) detectors to precisely pinpoint the location of the interaction of the X-ray with detector arrays and improve the timing resolution. In some embodiments, SPAD technology, particularly in conjunction with 3D electronics, allows arrays of small 30×30 μm₂ to 100×100 um² SPADs, thus leading to highly sensitive photodetectors with high timing resolution in the order of tens of picoseconds. The quality of the discrimination, along with the spatial resolution, can also be improved by using a magnification process such as increasing the distance between the volume of interest and the detector system. It will be appreciated by someone skilled in the art that the depth of interaction of scintillator-based detectors could have a lower spatial resolution for the depth of interaction (z coordinate) in embodiments where the time resolution averages about 10 picoseconds.

Scanning a person classified as suffering from obesity and/or that has a body mass index (BMI) equal to or greater than about 30 (bariatric patients) requires photons of higher energies than the standard energy range used in X-ray imaging (whether 2D or 3D). This will normally yield a lower contrast absorption image. Dynamic spatial reconstruction (DSR) is particularly useful for scanning bariatric patients since larger volumes generate more scatter noise that can be used for scatter image reconstruction. An optimization of the timing window may be beneficial to generate an absorption image with ballistic photons or scatter image with scattered photons.

Finally, embodiments of the invention can be well suited for gated imaging where the X-ray source could be turned on and off according to an external signal such as respiratory gating or cardiac to avoid motion artifacts and better visualize the organ. CNR could then be better improved in such circumstances since the organ motion can blur the acquired image.

It will be appreciated that any embodiment of the method described above may be implemented in a computing program product for generating an image of a region of interest from time-dependent X-photon detection data comprising a temporal point spread function (TPSF) distribution. The computing program product may include non-transitory memory storing executable program code that, when executed by a processor, causes said processor to perform any embodiment of the above methods. For example, the TPSF distribution of detected ballistic photons and scattered photons as a function of a corresponding time-of-flight for each detection location may be used to generate the image of the region of interest.

It will be appreciated that the method may be used by a system for X-ray imaging, which includes the memory 8 that stores the program code for performing the method proposed herein. A processor 7 to execute the program code stored in the memory 8 and perform the method steps. In one embodiment, the system may include a computing device 112 that includes both the processor 7 and the memory 8.

As illustrated in FIG. 12 one embodiment of the system 222 may include a pulsed X-ray imaging device 111 that acts as the acquisition device, a local workstation 113 (e.g., user interface, display, viewing station, a combination thereof, etc.), and the computing device 112.

As illustrated, the system 222 may optionally include a database 103 and/or an additional workstation 113. While the computing device 112 may be co-located with the acquisition device 111 and the local workstation 113, it will be appreciated that the computing device may be remotely located and connected to the acquisition device 111 over a data network, such as the Internet. The computing device 112 can thus receive from the X-ray imaging device the collected data representing the measured TPSF dataset over the data network and output the values of both the absorption coefficient and the at least one scattering coefficient for the voxels of the region of interest of the object for sending back to the workstation 113 or another remote workstation 101″ or a database 103.

The database 103 may be a secured database or cloud service for storing one or more of the outputs of the proposed method (e.g., the values of determined coefficients μₐ and μₛ for each voxel, visual representation generated therewith, any material or tissue information determined using the coefficients μₐ and μₛ, etc.), “raw” measured TPSF data, the predicted TPSFs or any other pertinent data used in or resulting from the proposed method.

It will be appreciated that, while some of the elements of the system 222 are shown as being located at various (e.g., remote) locations, they may all be located at a common location, some of the “remote” elements may share a same location, or only one of the elements (e.g., the remote computing device 112) may be located at a different location. In some embodiments, the various elements of the system 222 are in a same country or located in different countries.

In some embodiments, the user can send the instructions, commands, software updates or any data to the remote computing device 112 via the workstation 113.

What is claimed is:

1. An X-ray imaging apparatus comprising:
an input interface for receiving time-dependent X-photon detection data;
a processor; and
non-transitory memory storing program code that, when executed by said processor, causes said processor to:
receive said time-dependent X-photon detection data that comprises temporal point spread function (TPSF) distributions of detected ballistic photons and scattered photons as a function of a corresponding time-of-flight for each detection locations;
process said TPSF distribution for each of said detection locations to determine both a scatter coefficient and an absorption coefficient of X-photons for each voxel of a region of interest; and
generate an image of said region of interest using said scatter coefficient and said absorption coefficient of X-photons for each voxel of the region of interest.

2. The apparatus as defined in claim 1, wherein said program code causes said processor to:
use said TPSF distributions to generate an absorption representation and a scattering representation; and
to generate said image by using said absorption representation to correct said scattering representation.

3. The apparatus as defined in claim 1, wherein said program code causes said processor to generate a corrected scattering representation using a plurality of possible locations of a single scattering of said detected scattered photons and a detection location of said detected ballistic photons.

4. The apparatus as defined in claim 1, wherein said X-photon detection data comprises a plurality of time-of-flights and a corresponding detection location for each one of said time-of-flights.

5. The apparatus as defined in claim 1, wherein at least one pixel/voxel of said image is color coded as a function of an electron density or a corresponding material.

6. The apparatus as defined in claim 3, wherein said detected scattered photons are differentiated from said detected ballistic photons based on one of the group consisting of:
a time-of-flight threshold;
said time-of-flight threshold and a first time-of-flight cut-off; and
a shape of said TPSF.

7. The apparatus as defined in claim 1, wherein said identification of said detected ballistic photons from said time-of-flights comprises using at least one of:
a second time-of-flight cut-off; and
said shape of said distribution of said detected ballistic photons and said scattered photons as a function of a corresponding said time-of-flight.

8. The apparatus as defined in claim 1, wherein said program code causes said processor to further determine an electron density of at least one voxel of said image.

9. The apparatus as defined in claim 8, wherein said electron density is used to determine a corresponding material.

10. The apparatus as defined in claim 3, wherein said possible locations of said single scattering events are determined using back-projection, filtered back-projection, iterative method or artificial intelligence-based algorithms.

11. The apparatus as defined in claim 1, wherein said scatter coefficient and said absorption coefficient of the X-photons for each voxel of the region of interest are determined using back-projection, filtered back-projection, iterative or artificial intelligence-based algorithms.

12. A method of reconstructing a medical diagnostic image of a human patient, the method comprising:
receiving time-dependent X-photon detection data that comprises temporal point spread function (TPSF) distributions of detected ballistic photons and scattered photons as a function of a corresponding time-of-flight for each detection location;
using said TPSF distribution for each of said detection locations to determine both a scatter coefficient and an absorption coefficient of X-photons for each voxel of a region of interest; and
generating an image of said region of interest using said scatter coefficient and said absorption coefficient of X-photons for each voxel of the region of interest.

13. The method of claim 12, wherein using said TPSF distribution for each of said detection locations to determine the scatter coefficient comprises using said detection location and said time-of-flight of detected scattered photons having undergone a single scattering event to determine possible locations of said single scattering event of said detected scattered photons; wherein each one of said possible locations comprises at least one portion of an ellipsoid surface of possible positions of said single scattering event of a corresponding one of said detected scattered photons; and
wherein said generating said image comprises generating a scattering representation of said region of interest using at least a plurality of said possible locations.

14. The method as defined in claim 12, further comprising identifying said detected scattered photons having said single scattering event and said detected ballistic photons from said measurement based on at least one of:
a time-of-flight threshold;
said time-of-flight threshold and at least one time-of-flight cut-off; and
a shape of a distribution of said detected ballistic photons and said scattered photons as a function of a corresponding said time-of-flight.

15. The method as defined in claim 14, wherein said generation of said image comprises using said possible locations of said single scattering event and said detection locations of said detected ballistic photons to generate a corrected scattering representation.

16. The method as defined in claim 13, wherein said generation of said image further comprises:
using said detection locations of said detected ballistic photons to generate a scattering representation and an absorption representation, and
combining said scattering representation with said absorption representation to generate said image of said region of interest.

17. The method as defined in claim 13, wherein said possible locations of said single scattering event are determined using back-projection, filtered back projection, iterative or artificial intelligence-based algorithms.

18. The method as defined in claim 13, wherein a material of at least one voxel or pixel of said image is determined using said scatter coefficient and said absorption coefficient of X-photons.

19. A system comprising:
an X-ray imaging device comprising a pulsed X-ray source having a control signal; a time-sensitive X-ray detector having a time-dependent X-ray photon detection signal output; and circuitry, connected to said control signal and said time-dependent X-ray photon detection signal output, for collecting said data representing the measured TPSF dataset from a time-dependent X-ray photon detection signal of said time-sensitive X-ray detector;
a computing program product for generating an image of a region of interest from time-dependent X-photon detection data comprising temporal point spread function (TPSF) distributions of detected ballistic photons and scattered photons as a function of a corresponding time-of-flight for each detection location, the computing program product comprising non-transitory memory storing executable program code that, when executed by a processor, causes said processor to:
1) receive said time-dependent X-photon detection data;
2) process said TPSF distribution for each of said detection locations and measured energies to determine both a scatter coefficient and an absorption coefficient of X-photons for each voxel of a region of interest; and
3) generate an image of said region of interest using said scatter coefficient and said absorption coefficient of X-photons for each voxel of the region of interest;
a processor for receiving from the X-ray imaging device the collected time-dependent X-photon detection data, the processor being functionally connected to the memory of the computing program product for executing the program code to output the generated image of the region of interest; and
a workstation for receiving from the processor the outputted image of the region of interest.

20. The system of claim 19, wherein at least one of the computing program product, the processor, and the X-ray imaging device—is at a remote location from the workstation.
